(19) United States
(12) Patent Application Publication
(10) Pub. No.: US 2022/0165433 A1
(43) Pub. Date: May 26, 2022

(54) ASSESSMENT OF EXPOSURE TO AIRBORNE PATHOGENS

(71) Applicant: Signature Science, LLC, Austin, TX (US)

(72) Inventors: Brian Schimmoller, Charlottesville, VA (US); Molly Isbell, Charlottesville, VA (US)

(21) Appl. No.: 17/532,821

(22) Filed: Nov. 22, 2021

Related U.S. Application Data
(60) Provisional application No. 63/116,766, filed on Nov. 20, 2020.

(51) Int. Cl. G16H 50/80; A61B 5/00; G16H 50/20; G16H 50/50

(52) U.S. Cl. CPC G16H 50/80; G16H 50/50; G16H 50/20; A61B 5/00

(57) ABSTRACT

Technologies are provided for assessment of exposure to airborne aerosol pathogens (e.g., SARS-CoV-2). Exposure can be assessed using a model that yields a score quantifying comparative dose which can be classified into an exposure risk category in a particular scenario relative to a high-risk scenario. The model incorporates mechanistic, stochastic, and epidemiological factors that define the particular scenario within an activity region. The model can determine an average concentration of pathogen within the activity region, for a particular exposure duration, using an approach that includes near-field and far-field contributions. In some embodiments, an apparatus can access data from various devices. The data defines at least some of the factors the define the particular scenario. The apparatus can determine an average concentration within the activity region, and can cause a display device to present indicia indicative of the exposure risk.

### CROSS-REFERENCE TO RELATED APPLICATION

This application claims the benefit of and priority to U.S. Provisional Application No. 63/116,766, filed Nov. 20, 2020, the entire contents of which application are hereby incorporated herein by reference.

### BACKGROUND

Assessing worker and group risks associated with COVID-19 disease and SARS-CoV-2 exposure is now an everyday activity that organizations of all types must engage in. Those assessing exposures risks are regularly considering an array of factors using largely qualitative guidance from a variety of public health and media sources. Given the various factors that affect SARS-CoV-2 exposure, rapid qualitative assessments are particularly difficult when trying to compare various courses of action or potential mitigation options.

### SUMMARY

It is to be understood that both the following general description and the following detailed description are exemplary and explanatory only and are not restrictive. Methods and systems for determining exposure to airborne pathogens are described.

This disclosure recognizes and addresses, amongst other technical challenges, the issue of evaluating risk of exposure to airborne pathogens. To that end, the disclosure provides a quantitative tool that is based upon scientific principles and incorporates information regarding disease characteristics and pathogen behavior. As such, the disclosure can benefit risk assessors and decision-makers. Embodiments of this disclosure, individually or in combination, can assess exposure to airborne aerosol pathogens (e.g., SARS-CoV-2) addressing both near-field and far-field sources of pathogens by incorporating mechanistic, stochastic, and epidemiological factors that contribute to an inhalation dose. Factors considered in the model include: (1) the emission rate of the pathogen (e.g., virus, bacteria, phage, or fungus), (2) duration of exposure, (3) inhalation rates and exhalation rates based on activities of the group or individual, (4) ventilation rates (indoors and outdoors), (5) volume of indoor space, (6) filtration removal efficiency, (7) personal protective equipment (PPE) effectiveness, (8) social distancing, (9) group population's or individual's adherence to public health guidance, (10) size of the group, (11) prevalence of infection in the population or likelihood for an individual, (12) impact and prevalence of variants, (13) vaccination rates, and (14) pre-activity diagnostic testing strategies. Embodiments of this disclosure for determining exposure to airborne pathogens can access data from multiple devices (such as sensor devices, camera devices, server devices, and/or similar devices) where the data can be representative of an activity space and can permit determining values of one or more of the factors considered in the model. At least some of those devices can remotely located relative to a computing apparatus or computing system that applies the model to determine inhalation doses and risk scores for a group or each specific individual.

Embodiments of this disclosure, individually or in combination, provide a novel approach for estimating concentrations in the air of hazardous contaminants (such as virus-containing aerosols) resulting from the exhalation of people in close proximity (near-field contribution) and the concentration that builds up in the space or room over time (far-field contribution). Embodiments of the disclosure can be broadly applied to many situations (e.g., worker safety, the general public, and schools).

This summary is not intended to identify critical or essential features of the disclosure, but merely to summarize certain features and variations thereof. Other details and features will be described in the sections that follow.

### BRIEF DESCRIPTION OF THE DRAWINGS

The accompanying drawings, which are incorporated in and constitute a part of this specification, together with the description, serve to explain the principles of the methods and systems described.

FIG. 1 is a schematic diagram that summarizes various exposure factors that can determine inhalation dose and exposure risk to airborne pathogens in a model region, in accordance with one or more embodiments of this disclosure.

FIG. 2 illustrates an example of an operating environment for determination of inhalation dose and assessment of exposure risk to airborne pathogen, in accordance with one or more embodiments of this disclosure.

FIG. 2A is schematic block diagram of an example computing system for determination of inhalation dose and assessment of exposure risk to airborne pathogen, in accordance with one or more embodiments of this disclosure.

FIG. 3 illustrates an example of a graphical user interface, in accordance with one or more embodiments of this disclosure.

FIG. 4 schematically depicts another user interface, in accordance with one or more embodiments of this disclosure.

FIG. 4A illustrates a section of the user interface shown in FIG. 4.

FIG. 4B illustrates a section of the user interface shown in FIG. 4.

FIG. 4C illustrates a section of the user interface shown in FIG. 4.

FIG. 4D illustrates a section of the user interface shown in FIG. 4.

FIG. 4E illustrates a section of the user interface shown in FIG. 4.

FIG. 4F illustrates a section of the user interface shown in FIG. 4.

FIG. 5 is a schematic diagram of a well-mixed box (WMB) model.

FIG. 6 is a schematic diagram of a box-within-a-box model, in accordance with one or more embodiments of this disclosure.

FIG. 7 illustrates an example of an arrangement of a receptor and multiple sources within a grid of equidistant triangles used to partition an activity space, in accordance with one or more embodiments of this disclosure.

FIG. 8 illustrates an example of another arrangement of a receptor and multiple sources within a grid of equidistant triangles used to partition an activity space, in accordance with one or more embodiments of this disclosure.

FIG. 9 illustrates a receptor view within a grid of equidistant triangles used to partition an activity space, in accordance with one or more embodiments of this disclosure.

FIG. 10 shows a source view with two sources within a grid of equidistant triangles used to partition an activity space, in accordance with one or more embodiments of this disclosure.

FIG. 11A illustrates triangular prisms NF and FF within a grid of equilateral triangles used to partition an activity space, in accordance with one or more embodiments of this disclosure.

FIG. 11B illustrates triangular prisms NF and FF within a grid of equilateral triangles used to partition an activity space, in accordance with one or more embodiments of this disclosure.

FIG. 12 illustrates an example system for determination of inhalation dose and assessment of exposure risk to airborne pathogens, in accordance with one or more embodiments of this disclosure.

FIG. 13 illustrates an example training method for training a predictive model to determine inhalation dose and assessing exposure risk to airborne pathogens, in accordance with one or more embodiments of this disclosure.

FIG. 14 is a block diagram of a computing apparatus for implementing the determination of inhalation dose and assessing exposure risk to airborne pathogens, in accordance with one or more embodiments of this disclosure.

FIG. 15 is a flowchart illustrating an example method for determining inhalation dose and assessing exposure risk to airborne pathogens, in accordance with one or more embodiments of this disclosure.

### DETAILED DESCRIPTION

As used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another configuration includes from the one particular value and/or to the other particular value. When values are expressed as approximations, by use of the antecedent “about,” it will be understood that the particular value forms another configuration. It will be further understood that the endpoints of each of the ranges are significant both in relation to the other endpoint, and independently of the other endpoint.

“Optional” or “optionally” means that the subsequently described event or circumstance may or may not occur, and that the description includes cases where said event or circumstance occurs and cases where it does not.

Throughout the description and claims of this specification, the word “comprise” and variations of the word, such as “comprising” and “comprises,” means “including but not limited to,” and is not intended to exclude other components, integers or steps. “Exemplary” means “an example of” and is not intended to convey an indication of a preferred or ideal configuration. “Such as” is not used in a restrictive sense, but for explanatory purposes.

It is understood that when combinations, subsets, interactions, groups, etc. of components are described that, while specific reference of each various individual and collective combinations and permutations of these may not be explicitly described, each is specifically contemplated and described herein. This applies to all parts of this application including, but not limited to, steps in described methods. Thus, if there are a variety of additional steps that may be performed it is understood that each of these additional steps may be performed with any specific configuration or combination of configurations of the described methods.

As will be appreciated by one skilled in the art, hardware, software, or a combination of software and hardware may be implemented. Furthermore, a computer program product on a computer-readable storage medium (e.g., non-transitory) having processor-executable instructions (e.g., computer software) embodied in the storage medium. Any suitable computer-readable storage medium may be utilized including hard disks, CD-ROMs, optical storage devices, magnetic storage devices, memristors, Non-Volatile Random Access Memory (NVRAM), flash memory, or a combination thereof.

Throughout this application, reference is made to block diagrams and flowcharts. It will be understood that each block of the block diagrams and flowcharts, and combinations of blocks in the block diagrams and flowcharts, respectively, may be implemented by processor-executable instructions. These processor-executable instructions may be loaded onto a general-purpose computer, special purpose computer, or other programmable data processing apparatus to produce a machine, such that the processor-executable instructions which execute on the computer or other programmable data processing apparatus create a device for implementing the functions specified in the flowchart block or blocks.

These processor-executable instructions may also be stored in a computer-readable memory that may direct a computer or other programmable data processing apparatus to function in a particular manner, such that the processor-executable instructions stored in the computer-readable memory produce an article of manufacture including processor-executable instructions for implementing the function specified in the flowchart block or blocks. The processor-executable instructions may also be loaded onto a computer or other programmable data processing apparatus to cause a series of operational steps to be performed on the computer or other programmable apparatus to produce a computer-implemented process such that the processor-executable instructions that execute on the computer or other programmable apparatus provide steps for implementing the functions specified in the flowchart block or blocks.

Accordingly, blocks of the block diagrams and flowcharts support combinations of devices for performing the specified functions, combinations of steps for performing the specified functions and program instruction means for performing the specified functions. It will also be understood that each block of the block diagrams and flowcharts, and combinations of blocks in the block diagrams and flowcharts, may be implemented by special purpose hardware-based computer systems that perform the specified functions or steps, or combinations of special purpose hardware and computer instructions.

This detailed description may refer to a given entity performing some action. It should be understood that this language may in some cases mean that a system (e.g., a computer) owned and/or controlled by the given entity is actually performing the action.

This disclosure provide computing systems, computing devices, computer-implemented methods, computer-program products that, individually or in combination, may assess exposure to airborne aerosol pathogens (such as SARS-CoV-2) addressing both near-field and far-field sources of pathogens by incorporating mechanistic, epidemiological, and epidemiological factors that contribute to exposure dose. Factors considered in the model include (1) the emission rate of airbourne pathogen, (2) duration of exposure, (3) inhalation rates, (4) ventilation rates (indoors and outdoors), (5) volume of indoor space, (6) filtration removal efficiency, (7) PPE effectiveness, (8) social distancing, (9) group population's or individual's adherence to public health guidance, (10) size of the group, and (11) prevalence of infection in the population or likelihood for an individual, (12) impact and prevalence of variants, (13) vaccination rates, and (14) pre-activity diagnostic testing strategies.

Embodiments of this disclosure, individually or in combination, can be broadly applied to many situations (e.g., worker safety, the general public, and schools). In some cases, embodiments of this disclosure, individually or in combination, can provide a score indicative of risk of exposure for a group or specific individual to an airborne pathogen. The score can be aligned with the Occupational Safety and Health Administration (OSHA) classifications of exposure risks. In other cases, the score can be aligned with exposure risk classifications defined by other public health organizations (governmental or non-governmental) or experts. To that end, embodiments of this disclosure, individually or in combination, can determine a metric of exposure to the airborne pathogen in an particular scenario and can then compare that metric to a reference metric of exposure to the airborne pathogen in a scenario that is considered high risk by OSHA or other public health organizations (governmental or non-governmental) or experts.

Such metrics can quantify an average mass of inhaled airborne pathogen, and can be determined by applying an inhalation exposure dose model (also referred to as an exposure model) in accordance with aspects described herein. More specifically, embodiments of this disclosure provide a new approach for estimating concentrations in the air of hazardous contaminants (in this case, virus-containing aerosols) resulting from the exhalation of people in close proximity (near-field contribution) and the concentration that builds up in a confined space over time (far-field contribution).

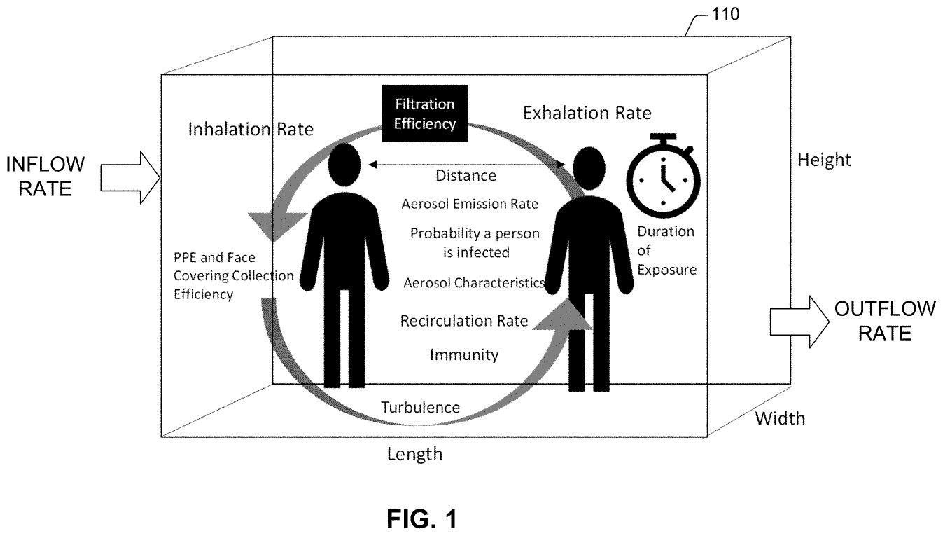

With reference to the drawings, FIG. 1 is a schematic diagram that summarizes various exposure factors that can determine exposure to an airborne pathogen in a model region 110, in accordance with one or more aspects of this disclosure. The exposure factors include mechanistic factors, stochastic factors, and epidemiological factors. Simply for purposes of illustration, Table 1 presents example of exposure factors and example ranges of magnitude for those factors.

TABLE 1
Examples of factors that affect inhalation dose and exposure risk, ranges thereof and
sources of data to may determine the value of the factors.

| | | | Example Range of |
|---|---|---|---|
| | | Basis and Potential | Magnitude in the |
| Factors | Type | Source of Input Data | Disclosure |
| Likelihood of | Stochastic | Obtained from data | Ranges over 5 orders of |
| infectious persons | | accessed via an | magnitude from the lowest |
| present in a group | | programming interface | (0.0001%) assumed for |
| | | (API), a user device, or | people adhering strictly to |
| | | other means. | public health guidance, to the |
| | | | highest (100%) for those |
| | | | known to be infected. |
| Number of persons in | Mechanistic/ | Obtained from outside | Ranges from 2 to 250 people, |
| the group | Stochastic | data accessed via API, | in some embodiments. The |
| | | sensors, a user device, | disclosure, however, is not |
| | | or other means | limited to that range, and |
| | | | there is no limit on the |
| | | | maximum number of persons |
| | | | in the group. |
| Distance between | Mechanistic | Obtained from sensor | Discrete options can be |
| people | | devices, a user device, | provided. For instance, 4.5 m |
| | | or other means | (~15 ft), 3 m (~10 ft), 2 m |
| | | | (~6 ft), 1 m (~3 ft), and 0.5 m |
| | | | (~1.5 ft). |
| Mask effectiveness | Mechanistic | Obtained from | Range of mask effectiveness |
| | | published sources or | values based on published |
| | | mask manufacturer's | data for cloth, surgical, and |
| | | data | N-95 masks. |
| Mask compliance on | Stochastic | Obtained from data | Ranges between 0 and 100 |
| the group | | accessed via API, sensor | percent. |
| | | devices, a user device, | |
| | | or other means | |
| Emission rate of | Mechanistic | Activity can be | Range of viral RNA |
| Infectious aerosols | | estimated by sensors | emissions rates by activity |
| released through | | (e.g, acoustic sensor | and intensity in viral quanta |
| respiration | | devices) | per hour. |
| Inhalation rate | Mechanistic | Activity can be | Typical inhalation rates for |
| | | estimated by sensors | adults at various activity |
| | | (e.g., acoustic sensor | intensities, between 3.6010⁻³ |
| | | devices) | m³ per min and 5.210⁻² m³ |
| | | | per min. |
| Duration of exposure | Mechanistic | Obtained from sensor | Varies between 5 minutes |
| | | devices, a user device or | and 12 hours |
| | | other means | |
| Indoors or outdoors | Mechanistic | Obtained from sensor | Indoor or Outdoor options |
| activity | | devices, a user device, | affect the form of the |
| | | or other means | concentration model used. |
| Ventilation rates (air | Mechanistic | Determined from data | Values can be based on |
| changes per hour | | obtained from sensor | published sources or |
| [ACH] or air exchange | | devices, a user device, | measurements. Range |
| rate [AER]) | | or other means | between 0.1 and 100 air |
| | | | changes per hour. |
| Aerosol settling rate | Mechanistic | Determined according | An additional quantity of |
| | | to various methods | 0.24 hr⁻¹ is added to the ACH |
| | | | for removal by deposition on |
| | | | surfaces. |
| Pathogen degradation | Mechanistic | Determined according | An additional quantity of |
| rate | | to various methods. The | 0.63 hr⁻¹ is added to the ACH |
| | | rate can be informed by | for pathogen degradation. |
| | | data obtained by | |
| | | temperature, humidity, | |
| | | or ultraviolet radiation | |
| | | sensor devices. | |
| Recirculating room | Mechanistic | Provided by system | Recirculation of filtered air |
| filtration rate and | | manufacturer or | assumed to occur at a rate of |
| removal efficiency | | installer. | 5 [L/s]/m² (1 cfm/ft²) or |
| | | | provided for the system by |
| | | | manufacturer or installer. |
| Volume of room or | Mechanistic | Obtained from sensor | Ceiling height ranges |
| activity space | | devices, a user device, | between 2.15 meters (7 feet) |
| | | or other means. | and 20 meters (65 feet). |
| | | | Room side dimensions range |
| | | | between 2 meters (7 feet) |
| | | | and 200 meters (650 |
| | | | feet). |
| Prevalence of COVID- | Epidemiological/ | Obtained from data | Active cases per 100,000 is |
| 19 in the community | Stochastic | accessed via API or | estimated by the published |
| | | other means. | “Average Daily Cases per |
| | | | 100,000 in the Last Week” |
| | | | available from various |
| | | | sources and estimates of the |
| | | | “Average Days Infectious” |
| | | | and “Undiagnosed Factor.” |
| Difference in the | Epidemiological | Determined from | Equivalent exposure dose |
| variants transmission | | published data | can be adjusted upward by a |
| rates versus wild type | | | factor proportional to the |
| pathogen | | | reported increased variant |
| | | | transmission. |
| Impact of | Epidemiological | Obtained from data | Immunities can be addressed |
| community's or | | accessed via API or | in two ways: (1) Reduced |
| group's immunity | | other means. | shedding (3 times reduction |
| from recovery and | | | can be used) (2) Input value |
| vaccination | | | of vaccine efficacy |
| | | | to function as “effective |
| | | | immunity barrier” at a level |
| | | | consistent with its published |
| | | | efficacy rate. |
| Impact of surveillance | Epidemiological | Determined from | Estimate |
| testing for the group | | published data | |

Exposure is defined as the contact of an airborne pathogen with an external boundary of a receptor (exposure surface) for a defined duration. Dose is the amount of material that passes through the boundary. In this disclosure, the boundary is the entrance to the respiratory system (e.g., through the nose and mouth). Inhalation dose is the amount of material that passes through the boundary of the respiratory system. The embodiments of this disclosure for exposure to airborne pathogens include a comparative dose and exposure risk model. This is because the embodiments of this disclosure incorporate the inhalation mechanism. Engaging in activities with high inhalation rates, such as group exercise or strenuous work tasks, can correlate with higher exposure risks and transmission risks.

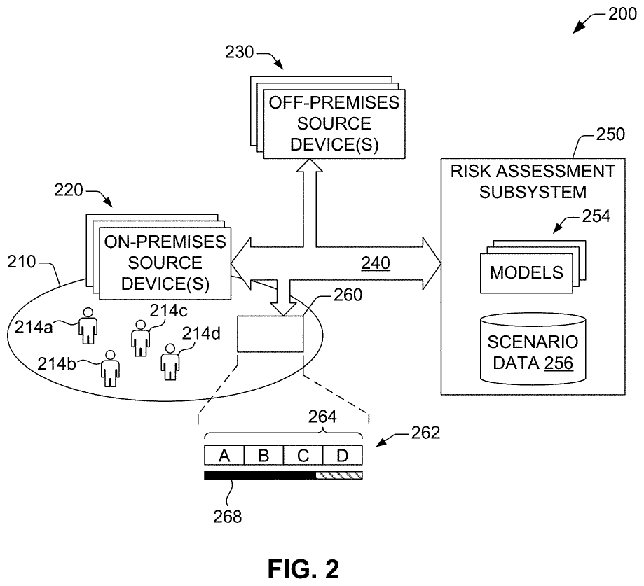

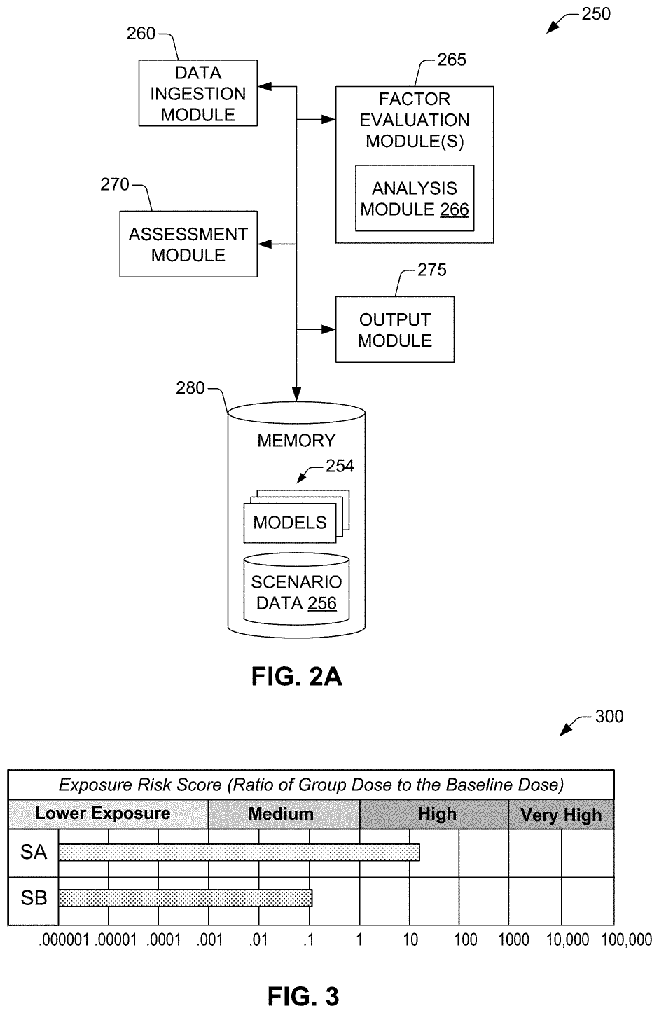

FIG. 2 illustrates an example of an operating environment 200 for assessment of inhalation dose and exposure risk to airborne pathogen, in accordance with one or more embodiments of this disclosure. The operating environment 200 includes a system having computing devices and/or other types of computing components. The assessment can be implemented for group or individual. As is described herein, the assessment can be based on a model of exposure to airborne pathogens. The model is referred to as an exposure model and incorporates multiple exposure factors. In some embodiments, the exposure model incorporates exposure factors of different types, such as mechanistic factors, stochastic factors, epidemiological factors. The exposure factors can be quantified in numerous ways and, after being quantified, the exposure factors define or otherwise configure a exposure scenario. The quantified exposure factors dictate, at least partially, a risk of exposure to an airborne pathogen within the exposure scenario.

A scenario can be specific to a region 210, an individual person, and/or a group of persons. The region 210 can be embodied in a confined space or a semi-open space. Examples of confined spaces include indoor spaces, such as classrooms, conference rooms, dining rooms, breakrooms, a business store, a childcare facility, a dogcare facility, and similar spaces. Semi-open spaces include one or several indoor spaces and one or several outdoor spaces. For the sake of illustration, the group of persons includes a first person 214a, a second person 214b, a third person 214c, and a fourth person 214d. As is noted in Table 1, the group of persons can include any number of persons ranging from two to 250, or even more in some cases, as there is no limit on the maximum.

Regardless of its type, as is illustrated in FIG. 2, the region 210 can include one or multiple on-premises source devices 220. Each one of the on-premises source device(s) 220 can generate data representative of a state of the region 210. Such data can permit quantifying one or several of the exposure factors incorporated into an exposure model in accordance with aspects of this disclosure. To that point, at least one of the on-premises source device(s) 220 can send the generated data to a risk assessment subsystem 250 via a communication architecture 240. The risk assessment subsystem 250 can determine a comparative inhalation dose and exposure risk for a group or specific individuals in accordance with aspects described herein. In some embodiments the communication architecture 240 include a network (e.g., a WiFi network, a public network, a private network, a cellular network, a combination thereof, or similar) and/or via any communication technique (e.g., a short-range communication technique, a long-range communication technique, etc.). Although shown as being external to the region 210, the risk assessment subsystem 250 can be present within the region 210 in some embodiments.

The on-premises source device(s) 220 can include, in some embodiments, multiple camera devices that can generate imaging data defining a stream of images, for example, of one or several areas of the region 210. The multiple camera devices can include stationary camera(s) affixed to a fixture within the region 210 and/or camera device(s) integrated into respective mobile devices present in the region 210. At least one of the multiple camera devices can send the imaging data to the risk assessment subsystem 250 via the communication architecture 240. The risk assessment subsystem 250 can then determine the dimensions of the area(s) by applying image processing techniques to imaging data received from one or multiple camera devices present in the region 210. The risk assessment subsystem 250 can use the dimensions of an area resulting from imaging data to estimate a volume of the area.

In one example, the region 210 can be an indoor space (e.g., a conference room or a classroom). A stationary camera device can be affixed to a wall in the indoor space and the second person 214b can have a mobile device (not depicted) that has a camera device integrated therein. Both the stationary camera and the camera integrated into the mobile device can generate respective imaging data and can send the imaging data to the risk assessment subsystem 250. The risk assessment subsystem 250 can then determine the dimensions of the indoor space by applying image processing techniques to the received imaging data. The risk assessment subsystem 250 can use those dimensions to estimate a volume of the indoor space.

Imaging data from camera device(s) within the on-premises source device(s) provides a wealth of other information besides dimensions of an area within the region 210. The risk assessment subsystem 250 can apply image processing techniques that include object recognition techniques. Hence, the risk assessment subsystem 250 can detect one or multiple objects from a stream of images defined by the imaging data. To that end, in some embodiments, as is shown in FIG. 2A, the risk assessment subsystem 250 can be configured with an analysis module 266 that can analyze the stream of images and extract data, features, and/or the like from the stream of images. In some cases, the analysis module can extract features comprising persons (e.g., a number of persons and positions of the persons in an area at specific times); fixtures within an area (e.g., air vents, doors, and so further); objects worn by persons within the area (e.g., particular clothing, masks, face shields, and/or other types of personal protective equipment, etc.); and/or the like. In some cases, the analysis module 266 can be part of one or more factor evaluation modules 265 (FIG. 2).

Accordingly, the risk assessment subsystem 250, via the analysis module 266 (FIG. 2), for example, can determine a number and locations of air vents within the region 210. In addition, or in some embodiments, the risk assessment subsystem 250 can determine a number and, in some cases, position of a group of persons and/or an individual person within the region 210. The number of persons and/or one or more respective positions can be determined at particular times over a span of time, for example. Further, or in yet other embodiments, the risk assessment subsystem 250 can determine a number of persons who are wearing masks or another type of PPE. As an example, the risk assessment subsystem 250 can determine a number and location of air vents within an indoor space, a number and position of a group of persons and/or individual person within the indoor space at a specific times over a span of time, and/or a number of persons who are wearing masks or another type of PPE.

Besides camera devices, or instead of camera devices, the on-premises source device(s) 220 can include sensor devices that can generate data representative of a state of the region 210. At least one of the sensor devices can send the data to the risk assessment subsystem 250 via the communication architecture 240. The sensor devices can form a homogenous group of sensor devices in that each one of the sensor devices probes a particular type of physicochemical property. In other cases, the sensor devices can form a heterogenous group of sensor devices in that a first sensor device in the group probes a first physicochemical property and a second sensor device in the group probes a second physicochemical property.

In some cases, a sensor device of the sensor devices can detect acoustic waves within the region 210. For instance, the sensor device can detect acoustic waves within an indoor space. The sensor device can be a stereographic microphone, for example. The sensor device or another device within the on-premises source device(s) 220 can send audio data representative of the acoustic waves to the risk assessment subsystem 250. In response to receiving the audio data, the risk assessment subsystem 250 can determine one or multiple attributes of the detected acoustic waves. For example, the risk assessment subsystem 250 can determine volume (or amplitude) or the acoustic waves. Using that volume, the risk assessment subsystem 250 can generate an estimate of emission rate of shed aerosols containing infectious pathogens. The risk assessment subsystem 250 can generate that estimate using the relationship of the overall noise level, the number of people, and the shedding rate associated with various vocalization intensities (e.g., talking, shouting, and singing). In some embodiments, as is illustrated in FIG. 2A, the risk assessment subsystem 250 (FIG. 2) can include one or multiple factor evaluation modules 265. One of the factor evaluation model(s) 265 can determine such an estimate.

In addition, or in other embodiments, the sensor devices can include a first sensor device that probes amount of carbon dioxide (CO₂) within the region 210, and one or several second sensor devices or camera devices that can detect the number of persons within the region 210. The first sensor device, the second sensor device(s), and/or camera devices also can detect a a time interval during which a person or persons have been present in the region 210. The first sensor device and at least one of the second sensor device(s) can send data indicative of measurements from both first sensor device and the second sensor device(s) to the risk assessment subsystem 250. In response of receiving such data, the risk assessment subsystem 250 can generate an estimate of air change rate within the region 210.

Detection of the number of persons within the region 210 also can be accomplished using one or multiple sensor devices. For instance, in some embodiments, the sensor devices in the on-premises source device(s) 220 can include a sensor device that can detect the positions of devices that emit wireless signals at various points in time. For example, the sensor device can detect wireless pilot signals emitted according to one or more radio technology protocols. In one example, the sensor device can detect wireless pilot signals emitted according to a first low-power radio technology protocol (such as Bluetooth™) or a second low-power radio technology protocol (Wi-Fi protocols, for example). In some cases, the sensor device can detect both of such types of wireless pilot signals. In other cases, a first sensor device can detect a first type of wireless pilot signals and a second sensor device can detect a second type of wireless pilot signals. Such sensor devices can determine unique identifiers carried by the pilot signals and can send the unique IDs to the risk assessment subsystem 250. In response to the receiving the unique IDs, the risk assessment subsystem 250 can generate a count of different unique IDs and can assign the count to the number of persons within the region 210. In some embodiments, one of the factor evaluation model(s) 265 (FIG. 2) can determine the number of persons in such a fashion.

Further, or in some embodiments, the sensor devices can include temperature sensor devices, humidity sensor devices, and ultraviolet (UV) radiation intensity that can generate data that can be used to estimate a rate of degradation of a contaminant (such as virus-containing aerosols). To that end, one or more of such sensor devices can send the data to the risk assessment subsystem 250 via the communication architecture 240. In response to receiving such data, the risk assessment subsystem 250 can generate an estimate of the rate of degradation of the contaminant. In some embodiments, one of the factor evaluation model(s) 265 (FIG. 2) can determine such an estimate.

In some embodiments, the on-premises source device(s) 220 can include one or multiple sensor devices that can measure instantaneous air speed and direction. Such sensor device(s) can generate data that can be used to estimate turbulence in the region 210 (e.g., in an indoor space or an outdoor space). At least one of such sensor(s), or another one of the on-premises source device(s) 220, can send the data to the risk assessment subsystem 250. The risk assessment subsystem 250 can receive the data to generate an estimate of eddy diffusivity which is used to determine concentration of disease-containing aerosols in the region 210. In some embodiments, one of the factor evaluation model(s) 265 (FIG. 2) can determine such an estimate.

As is illustrated in FIG. 2, the risk assessment subsystem 250 also can access various types of data from one or multiple off-premises source device(s) 230. Such data can define one or multiple factors that can dictate, at least partially, exposure to an airborne pathogen. In some embodiments, as is illustrated in the computing system 250 shown in FIG. 2A, to access that data, the risk assessment subsystem 250 can include an ingestion module 260 that can access the data. In one example, the ingestion module 260 can be embodied in, or can constitute, a server device that provides application programming interfaces (APIs) that can be used to obtain data from the off-premises source device(s) 230.

In some embodiments, the off-premises source device(s) 230 can include multiple server devices. A group of the multiple server devices can provide, or otherwise make available, geolocation data. The risk assessment subsystem 250, via the data ingestion module 260 (FIG. 2), for example, can access the geolocation data via an API interface. In some cases, the risk assessment subsystem 250 can generate an estimate positions of respective one or more persons within the region 210, e.g., an indoor space and/or an outdoor event. In some embodiments, one of the factor evaluation model(s) 265 (FIG. 2) can generate the estimate of position of respective person(s) within the region 210.

In addition, or in other embodiments, a second group of the multiple server devices can provide, or otherwise make available, data indicative of disease prevalence and/or a variant of a disease's prevalence. In one example, the second group of server devices can host a government website or another of website. The risk assessment subsystem 250, via the data ingestion module 260 (FIG. 2), for example, can access such disease data via one or multiple APIs. The risk assessment subsystem 250 can generate, using the disease data, an estimate of the number of cases among a group in an indoor space or at an outdoor event. In some embodiments, one of the factor evaluation model(s) 265 (FIG. 2) can determine such an estimate.

Further, or in another example, the second group of the multiple server devices also can provide, or otherwise make available, immunity data indicative of vaccination prevalence and/or second immunity data indicative of counts of recovered cases. The risk assessment subsystem 250, via the ingestion module 260 (FIG. 2), for example, can access such disease data via one or multiple APIs. The risk assessment subsystem 250 can generate, using one or both of the immunity data or the second immunity data, an estimate of a number of people with immunities to an infectious disease among a group of persons within the region 210, e.g., an indoor space and/or at an outdoor event. It is noted that immunity data can be applicable to a population in a location that contains the region 210. Thus, the immunity data can be representative of the group of persons within the region 210. In one example, immunity data available Fairfax County, VA, can be applied to people in a restaurant in Fairfax County, VA. In some embodiments, one of the factor evaluation model(s) 265 (FIG. 2) can determine such an estimate of the number of persons.

In response to accessing data defining factors that define an exposure scenario for the group of persons or individual person within the region 210, the risk assessment subsystem 250 can retain at least a portion of such data or an aggregated version thereof, as scenario data 256 within one or more memory devices. The risk assessment subsystem 250 also can determine an inhalation dose within the region 210 for the group of persons or individual persons, to include each specific persons individual accumulated inhalation dose based upon their positions over time. To that end, the risk assessment subsystem 250 can include one or multiple models 254. The multiple models 254 can include an inhalation dose model (also referred to as exposure model) for the airborne pathogen and a exposure risk scoring model for the group of persons or individual person, to include each specific person's individual accumulated exposure risk based upon their positions over time. The exposure risk scoring model can yield a risk score ε that is the ratio of inhalation doses quantifying a comparative inhalation dose and providing a value to categorize within a defined exposure risk band for risk of exposure to the airborne pathogen within the region 210 for the group of persons or individual person, to include each specific person based upon their positions over time. As is described herein, the risk score ε comprises a ratio of the inhalation dose and a baseline inhalation dose. In some embodiments, the risk assessment subsystem 250 can include an assessment module 270 (FIG. 2) that can determine inhalation doses by applying the inhalation dose model to the data defining such factors. The assessment module 270 (FIG. 2) also can generate risk scores using the exposure risk model.

The baseline inhalation dose also can be determined using the exposure model on a high-risk baseline scenario that corresponds, in some embodiments, to a scenario that is deemed high-risk by a public health organization (governmental or non-governmental) (e.g., OSHA, Centers for Disease Control and Prevention (CDC), a health department, or a medical authority). By comparing the exposure dose calculations to such a baseline scenario, results of the exposure risk model scoring model of this disclosure can be aligned with authority-defined risk classifications of exposure risks.

In some embodiments, the high-risk baseline scenario can be defined to represent a person (e.g., medical worker) who is exposed to a person infected with airborne infectious disease. Table 2 presents an example definition of a high-risk baseline scenario in terms of the multiple factors contemplated in the exposure risk scoring model of this disclosure. The exposure risk scoring model of this disclosure is then applied to the defined high-risk baseline scenario to (baseline generate the baseline value E_{Group Dose}^{(baseline)} Numerical inhalation dose values for other scenarios are compared to that baseline value through the foregoing ratio.

TABLE 2
Example exposure factors corresponding to high-risk baseline scenario.

| Factors considered in the Inhalation Dose | |
|---|---|
| Determination for High-Risk BL Scenario | Values Defining the High-Risk BL Scenario |
| Likelihood of Infectious persons present in the | 100% known infection assumed in the room. |
| group | |
| Number of persons in the group | 2 persons. |
| Distance between people | Approximately 1 m, based on estimated average |
| | distance during the event that is less than 6 feet |
| Mask effectiveness | No masks are worn |
| Mask compliance on the group | No masks are worn |
| Emission rate of Infectious aerosols released | 11.4 quanta per hour, associated with speaking |
| through respiration | while standing. |
| Inhalation rate | 13 liters per min (0.46 cfm) based on |
| | assumption of light intensity |
| Duration of exposure | 15 minutes, wich can be considered as a |
| | significant exposure duration |
| Indoors or outdoors activity | Indoor |
| Ventilation rates (air changes per hour [ACH] | 6 ACH based upon CDC guidance for health |
| or air exchange rate [AER]) | care facilities. |
| Aerosol settling rate | Add 0.24 to the ACH for the removal by |
| | deposition on surfaces. |
| Pathogen degradation rate | Add 0.63 to the ACH for the viral aerosol |
| | degradation assuming 20.1 degrees C. and 38% |
| | humidity |
| Recirculating room filtration rate and removal | No additional contribution to ACH from |
| efficiency | recirculating air filtration is assumed. |
| Volume of room or activity space | Room size assumed to be a small examination |
| | room that was 9 square meters (96.8 square feet) |
| | with a ceiling height of 2.74 meter (9 feet) |
| Prevalence of COVID-19 in the community | Since the likelihood of infectious persons present |
| | in the group is 100%, the prevalence of COVID- |
| | 19 in the community is not used in the exposure |
| | risk scoring model for this high-risk BL |
| | scenario. |
| Difference in the variants transmission rates | The wild type pathogen is assumed. |
| versus wild type pathogen. | Accordingly, no adjustment is introduced. |
| Impact of community's or group's immunity | No immunity is assumed. Accordingly, no |
| from recovery and vaccination | adjustment is introduced. |

In response to determining the risk score ε, the risk assessment subsystem 250 can cause or otherwise direct a display device 260 to present indicia indicative of the risk score ε. As is illustrated in FIG. 2, the display device 260 can be present within the region 210. One or more persons within the region 210 can view the display device 260. In some embodiments, the display device 260 can be embodied in an LED display device, and can be affixed to a fixture within the region 210. In other embodiments, the display device 260 can be part of a mobile device operated by a person within the region 210. For example, the region 210 can be a conference room and that person can be an office manager that has organized an activity (e.g., a colloquium) in the conference room. Regardless of its particular embodiment, by presenting the indicia indicative of the risk score ε, the display device 260 can provide actionable information that can be used to mitigate, or entirely avoid, a pathogen spreader event. In some embodiments, the risk assessment subsystem 250 can include an output module 275 (FIG. 2) that can cause or otherwise direct the display device 260 to present the indicia indicative of the risk score ε.

Indicia 262 shown in FIG. 2 constitute an example of the indicia indicative of the risk score ε. The indicia 262 includes a first visual element 268 indicative of a magnitude of the risk score ε (black section), and a second visual element 264. The second visual element 264 identifies four risk categories denoted by A, B, C, and D (simply for the sake of nomenclature). Each one of those categories can be mapped to a respective range of ε.

The interface 300 shown in FIG. 3 includes another example of indicia indicative of the risk score ε. The interface 300 includes indicia for two risk scores corresponding to a first scenario (denoted by “SA”) and a second scenario (denoted by “SB”). The two scenarios can correspond to respective configurations of the exposure factors. As is illustrated in FIG. 3, the risk score can be categorized in four risk bins: “Lower Exposure,” “Medium,” “High,” and “Very High.”

With further reference to FIG. 2, in some embodiments, the risk assessment subsystem 250 can access data defining exposure factors as the data becomes available at a source device. For instance, the data can be accessed in nearly realtime. Using the available data, the risk assessment subsystem 250 also can determine a risk score ε as the data becomes available, thus generating updated risk scores as a function of time. The risk assessment subsystem 250 can then cause the display device 260 to present the updated risk scores as those scores become available. As such, in some cases, the display device 260 can serve as a monitoring unit for exposure risk at the region 210.

In some embodiments, the risk assessment subsystem 250 can generate a file containing various data and metadata that summarize one or multiple exposure scenarios. At least one of those exposure scenarios can be previously configured using data from the on-premises source device(s) 220 and/or the off-premises source device(s) 230. The file can be formatted in several ways and can be consumed by a document processing application, for example, retained within a user device (such as a mobile device operated by one of the persons in the region 210) or another type of computing device. Consumption of such a configuration file can cause a computing device to present a graphical user interface (GUI) containing multiple UI elements. The multiple UI elements can describe attributes that define an exposure scenario. The exposure scenario can be described individually or in juxtaposition with another exposure scenario described by multiple second UI elements. The GUI that is presented also can include indicia indicative of one or more risk scores. In some cases, the GUI also can include indicia defining a recommendation to reduce exposure in scenarios where a risk score exceeds a defined threshold score.

In some embodiments, the GUI also can include selectable UI elements that, in response to being selected, can permit receiving input data to update an exposure scenario described in the GUI. Input data received via the GUI can be sent to the risk assessment subsystem 250. In response to receiving such input data, and, in some cases, using extant data, the risk assessment subsystem 250 can generate an updated risk score ε for the updated exposure scenario. The risk assessment subsystem 250 can cause the GUI to be updated to present the updated risk score, for example.

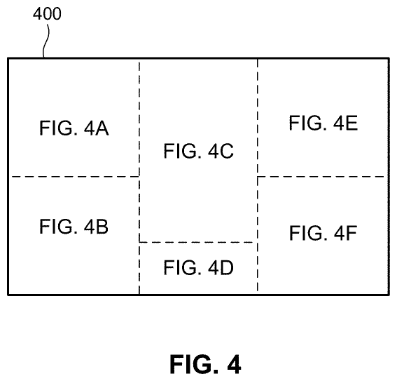

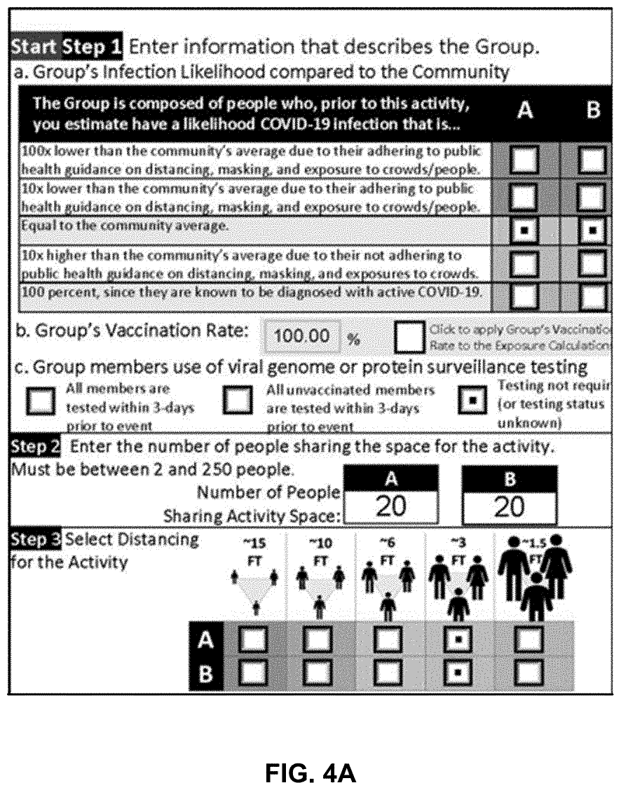

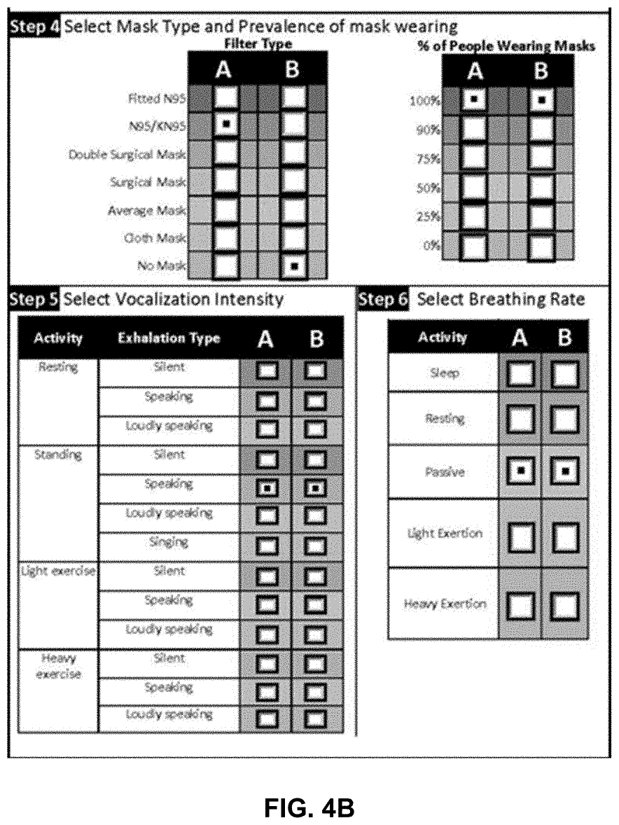

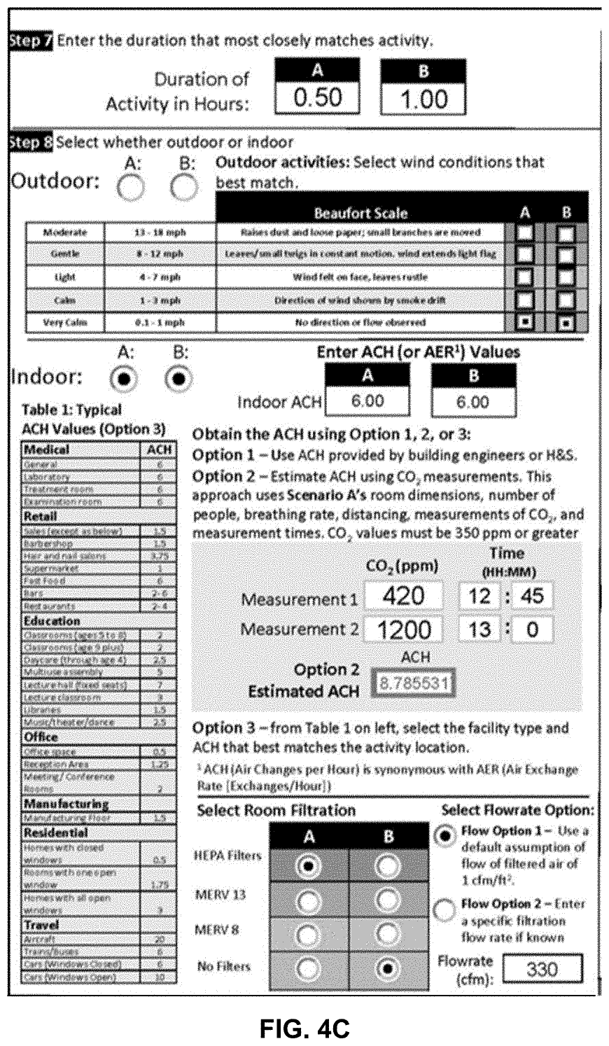

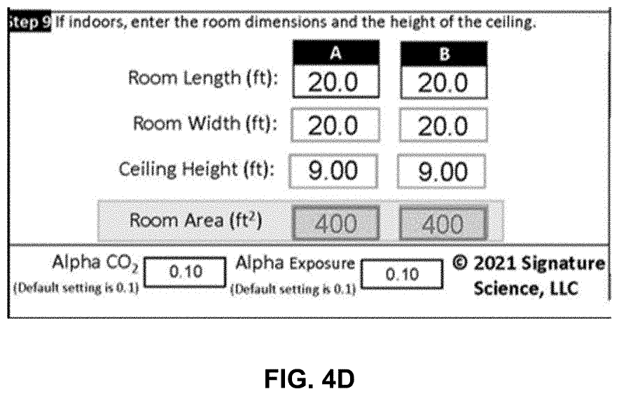

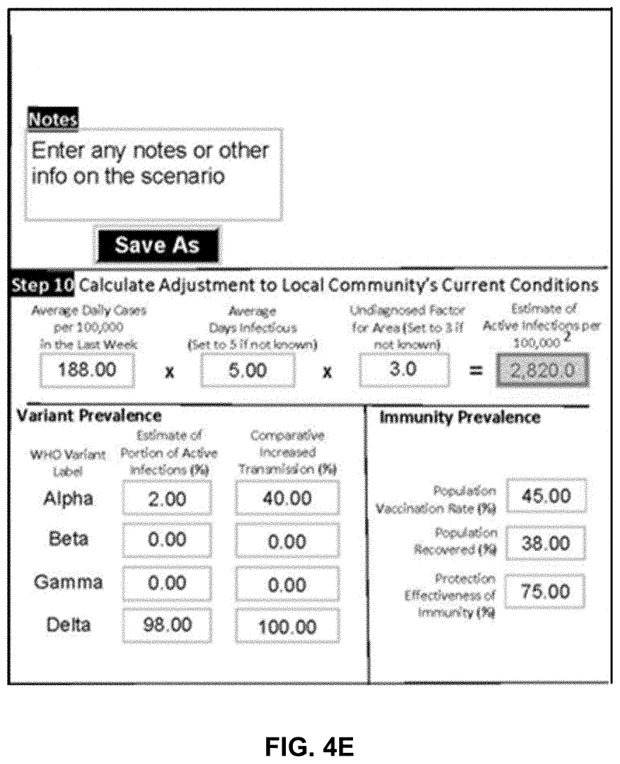

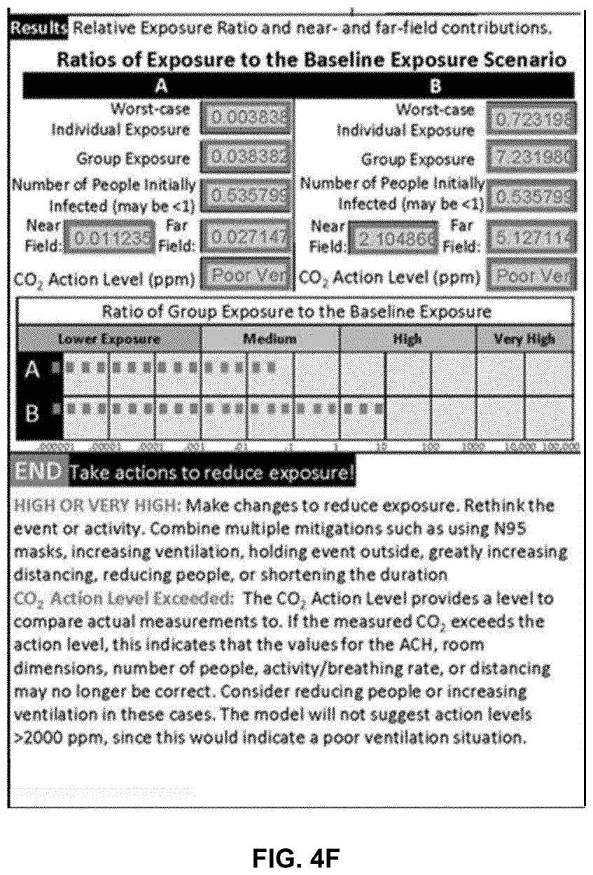

FIG. 4 schematically depicts an example of a UI 400 that can embody the GUI that summarizes one or more embodiments in accordance with aspects described hereinbefore. Additionally, the portion of the UI 400 shown in FIG. 4A, FIG. 4B, FIG. 4C, FIG. 4D, FIG. 4E, FIG. 4F illustrate respective sections of UI 400 depicted in FIG. 4.

The starting point for the mechanistic inhalation dose model (also referred to as exposure model) is to use the relationship that defines group-wide inhalation dose as a linear system where

E_{Group Dose}=C̅×Q̅_{Inhalation}×Δt×Pe_{Exposed}

where Q̅_{Inhalation} is the average inhalation rate for the group, C̅ is the average concentration of the agent (in this case, aerosols containing an airborne pathogen), Δt is the duration of group exposure, and Pe_{Exposed} is the number of people exposed in the group. The E_{Group Dose} represents the total mass of contaminant that enters the respiratory tracts of all of the group by inhalation over the duration Δt of the potential exposure or event. The fate or dynamics of the pathogen within the respiratory tract are not considered in the model and it would be part of a transmission process.

The exposure risk scoring model of this disclosure can take the form of a relative exposure dose model that compares a specific evaluated scenario to a defined high-risk baseline (BL) scenario by using the following ratio:

$$ɛ = \frac{{E}_{Group Dose}^{( evaluated )}}{{E}_{Group Dose}^{( baseline )}}$$

Thus, the ratio of exposure dose is equal to 1 when the specific scenario results in a group-wide inhalation dose having a value that is equal to the value of the group-wide inhalation dose corresponding to high-risk baseline scenario, the ratio is 1. The ratio may be orders of magnitude greater or less than 1 depending on the specific evaluated scenario. Such a comparative dose approach is advantageous since it allows the predictive model to compare exposure doses for other scenarios to a dose that represents a high risk that workers or group members would be recommended to avoid by authorities or experts.

One of the elements that the exposure risk scoring model estimates is the concentration of pathogen-containing aerosols that results from exhalation (e.g., breathing, speaking, coughing, singing, or similar) from people who are in close proximity. To that end, embodiments of this disclosure provide a concentration model that can assess both the contributions of concentration of pathogen due to the “nearness” of people (that is, people in the “near field” whether indoors or outdoors) and the buildup of concentration in a room over time (that is, a “far field” exposure contribution). Indoors, the far-field exposure contribution is approximately equivalent to that provided by a well-mixed box (WMB) assumption. Outdoors only the near-field concentration contributions are used because the far-field contribution is considered to be negligible. The final result is the sum of the near-field exposure and far-field exposure contributions. As is disclosed herein, the concentration model of this disclosure extends the near-field and far-field approach to groups of people at set distancing intervals by applying the superposition principle and using an eddy diffusivity calculation that is a function of air change rate within a space and the size of the space.

The concentration model relies on a stochastic approach that estimates the number of infections in a group of people. The number of infections is correlated to the quantity of pathogen-containing aerosols emitted in the modeled scenarios. In the defined high-risk baseline scenario, the group of people has two persons (see Table 2) and one infected person is assumed to be in that group. For an evaluated scenario, the number of infections in the group can be dependent on an estimate of the group's behavior characteristics and an estimate of the number of active cases in the community population, for example. Such a number of active cases can be determined by the product of the prevalence of diagnosed pathogen-induced infections in the community, an estimate of the duration of infectiousness, and an estimate of the fraction of cases thought to be undiagnosed. The resulting number of active infections may be less than or greater than 1.

The exposure risk scoring model can adjust a calculated exposure dose ratio by additional factors, such as (1) concentration of pathogen-containing aerosols that occurs as a result of the exhalation from people in close proximity; (2) number of infections in the group; (3) current community prevalence of variants; (4) relative infectiousness of the prevalent variants; (5) current prevalence of immunity in the community of group gained by recovery or vaccination; (6) efficacy of immunity in preventing transmission; and (7) efficacy of surveillance testing of the group. The risk assessment subsystem 250 can assess two scenarios, side-by-side. Additionally, embodiments of the disclosure can present results for the worst-case individual exposure, total group exposure, and near-field and far-field contribution to the total group exposure.

As is described hereinbefore, embodiments of the disclosure can provide an actionable risk score by categorizing final relative exposure dose ratios into four bins, for example, ranging from “Lower Exposure” through “Very High Exposure.” In some embodiments, the risk score can be presented graphically (see FIG. 2, for example).

As mentioned, the starting point for the mechanistic inhalation exposure dose model is to use the relationship that defines group-wide inhalation dose as a linear system where:

E_{Group Dose}=C̅×Q̅_{Inhalation}×Δt×Pe_{Exposed} Eq. (1)

There are variety of ways of estimating concentration of contaminants in the air. Several commonly used methods include WMB models, computational fluid dynamic (CFD) models and Gaussian dispersion models. Computational fluid dynamics based models use numerical solutions of the advection dispersion equation (ADE) that are tailored to the specific geometry, scale and temporal lengths, and flow regimes and are capable of modeling the complexities of particle dynamics, inhalation, exhalation, and interaction with flows in a building. Gaussian models use an explicit solution of the ADE, and are, therefore, computationally fast compared to CFD models. The WMB model is the simplest model that can be used to estimate concentrations of contaminants in air. The WMB model treats a room as if it were a continuous stirred-tank reactor (CSTR) and uses the basic equations for concentration that were developed for modeling continuous reactors in chemical engineering.

The basic equation for the single WMB model is the following:

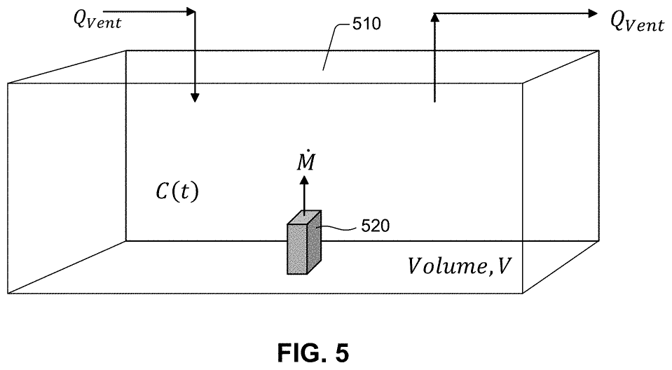

Vdt=\dot{M}dt−QᵥₑₙₜCdt Eq. (2)

where V is the volume of the box, Qᵥₑₙₜ is the ventilation rate (in units of volume per time) through the box, \dot{M} is the emission rate (in units of mass per time), and C=C(t) is the instantaneous (or time dependent) m agent concentration. FIG. 5 illustrates an example of a WMB model. As is illustrated in FIG. 5, the WMB model considers a single zone defined by a surface bounding a region 510 having volume V. The single zone encompasses a pathogent emitter 520.

In some configurations, it is assumed that the emission rate \dot{M} is constant starting at a time defined as zero, the time-varying Eq. (2) takes the form:

$$C ( t ) = \frac{\dot{M}}{{Q}_{Vent}} ( 1 - {e}^{- \frac{{Q}_{vent}}{V} t} ) . \qquad Eq . ( 3 )$$

After enough time has passed to achieve equilibrium, the concentration model takes the following form:

$${\overline{C}}_{eq} = \frac{\dot{M}}{{Q}_{Vent}} . \qquad Eq . ( 4 )$$

The foregoing equilibrium expression (Eq. (3)) can be adopted in scenarios where overestimating an actual concentration of a pathogen might be acceptable. For instance, once the value of

$$\frac{{Q}_{vent}}{V} t$$

t is 3 or higher, the value of C(t) is 95 percent of the equilibrium concentration. In some cases, a value of

$$\frac{{Q}_{vent}}{V} t \geq 3$$

may occur in a just a few minutes, especially in scenarios where the box volume is small and ventilation rates are large. In many cases, 95% of the equilibrium concentration would be achieved in under 30 minutes. Accordingly, while conservative, the equilibrium approximation may be valid in many exposure scenarios.

Because a conventional WMB model has a single zone and treats a contaminant as being instantaneously completely mixed throughout a volume of air, such a conventional WMB model yields the same exposure irrespective of how close or far people are located within the single zone.

### Near Field (NF)/Far Field (FF) Two-Box Model

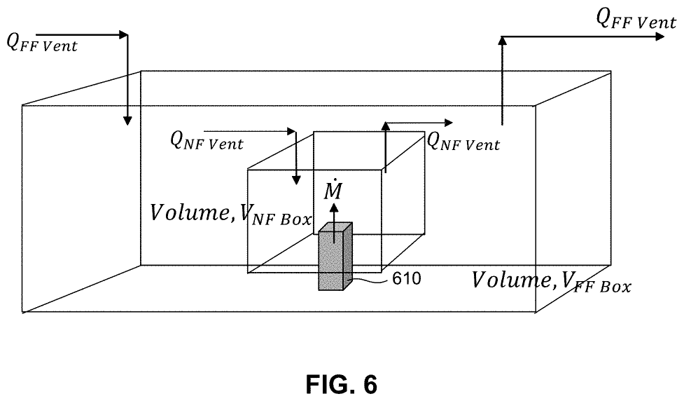

In the field of industrial hygiene, it is recognized that the single-zone box model may underestimate exposures experienced by receptors (e.g., people) close to a hazard, since it assumes that the concentration is instantaneously well-mixed over the volume of the space (e.g., a room) containing the receptors. To address the issue of estimation of high concentrations near a source, a “box within a box” model can be leveraged or otherwise relied upon. In that model, an inner box or near-field (NF) box contains a contaminant source and a receptor, and a larger box, or far-field (FF) box, encompasses the NF box and represents the entire volume of a space (e.g., a room). The outer box concentration can be thought of as the background concentration for the inner box concentration. FIG. 6 illustrates a “box-within-a-box” model that can provide localized higher concentration in the vicinity of a source 610. The approach may be based on and/or associated with imaging data, for example, received from a camera device within the on-premises source device(s) 220.

The time-dependent concentration at the receptor can be estimated by adding the NF concentration contribution and the FF concentration contribution:

$$C ( t ) = {C ( t )}_{NF} + {C ( t )}_{FF} = \frac{\dot{M}}{{Q}_{N F}} ( 1 - {e}^{- \frac{{Q}_{N F}}{{V}_{N F}} Δ t} ) + \frac{\dot{M}}{{Q}_{F F}} ( 1 - {e}^{- \frac{{Q}_{F F}}{{V}_{F F}} Δ t} ) \qquad Eq . ( 5 )$$

where, M is the continuous mass release rate of the contaminant of concern, Q_{NF} is the NF volumetric flow rate, Q_{FF} is the FF volumetric flow rate, V_{NF} is the NF volume, V_{FF} is the FF volume (or volume of the room or activity space), and Δt is the elapsed time since the start of the release. In some configurations, \dot{M} can be expressed in units of mass per minute; both Q_{NF} and Q_{FF} can be expressed in m³ per minute; both V_{NF} and V_{FF} can be expressed in (m³), and Δt can be expressed in minutes.

Assuming both boxes to be at equilibrium, Eq. (5) has the following form:

$${\overline{C}}_{eq} = {C}_{eq , NF} + {C}_{eq , FF} = \frac{\dot{M}}{{Q}_{NF}} + \frac{\dot{M}}{{Q}_{FF}} \qquad Eq . ( 6 )$$

The foregoing equation can be expressed in terms of appropriate ACHs (commonly used measures of the ventilation in buildings). Specifically, by adopting the minute as unit of time and expressing the ventilation rates Q_{NF} and Q_{FF} using appropriate respective volumes (V_{NF} and V_{FF}) and appropriate air change rates, Eq. (6) can be expressed as:

$${\overline{C}}_{eq} = \frac{\dot{M}}{{V}_{NF} \frac{{ACH}_{NF}}{60}} + \frac{\dot{M}}{{V}_{FF} \frac{{ACH}_{F F}}{60}} \qquad Eq . ( 7 )$$

where ACH_{NF} is the NF air change rate (hr⁻¹) and ACH_{FF} is the FF air change rate (hr⁻¹).

For the time-dependent form of the concentration can be expressed as follows (since the volumes in the exponential term cancel themselves out):

$$C ( t ) = \frac{\dot{M}}{{V}_{NF} \frac{{ACH}_{NF}}{60}} ( 1 - {e}^{- \frac{{ACH}_{NF}}{60} Δ t} ) + \frac{\dot{M}}{{V}_{FF} \frac{{ACH}_{FF}}{60}} ( 1 - {e}^{- \frac{{ACH}_{FF}}{60} Δ t} ) \qquad Eq . ( 8 )$$

In some embodiments, the NF flow rate Q_{NF} can be equal to ½×s×FSA, where FSA is the free surface area of the assumed NF control volume, and s is a random air speed (instantaneous in random direction) at the interface of the NF and FF zones. The ½ factor is assumed because, in order to ensure conservation of mass, half of the air volume is entering the control volume and half of the air is leaving the control volume. Further, in some configurations, s can be equal to 15.1 meters per minute (50 feet per minute) when strong air currents are present. In addition, or in other configurations, s can be equal to 3.0 meters per minute (10 feet per minute) when air currents are lacking near the NF zone. It yet other configurations, S can be equal to the median random air speed for indoor office and home spaces, which median speed has been observed to have a magnitude between 0.05 and 0.1 meters per sec. In still other configurations, s can be equal to 0.06 meters per second (3.6 meters per minute) with the FSA approach in indoor settings. Accordingly, ACH_{NF} can be calculated using the FSA approach as follows:

$${ACH}_{NF} = \frac{\frac{1}{2} \times s \times FSA \times 60}{{V}_{NF}} \qquad Eq . ( 9 )$$

Rather than using commonplace values of s, embodiments of the disclosure determine ACH_{NF} using an effective s_{eff} that varies with distance from the source and is derived from an estimate of the eddy diffusivity. To that end, the NF/FF two-box model in accordance with aspects of this disclosure uses eddy diffusivity instead of the random air speed s in order to provide the mixing dynamics. More specifically, a continuous point release is assumed and a concentration model is implemented using both a first NF/FF two-box model having a spherical NF volume, and a second NF/FF two-box model having a hexagonal prism NF. Table 3 presents expressions for equilibrium concentration are shown for such concentration models. As a further comparison, Table 3 also presents a pathogen concentration profile in equilibrium C(x) as a function of position relative to the pathogen source, where C(x) is obtained using a Gaussian solution of a dispersion equation when there is no advection (e.g., mean wind speed is equal to zero).

TABLE 3
Expressions for equilibrium concentration for various models.

| | NF/FF two-box model with | NF/FF two-box model with |
|---|---|---|
| Gaussian solution with a | a spherical prism as a NF | hexagonal prism as a NF |
| source above a surface | volume | volume |
| $\begin{matrix}C ( x ) = \frac{M}{2 π K x} , \\ where x is distance from the \\ source and K is a constant \\ that characterizes diffusion of \\ aerosol .\end{matrix}$ | $\begin{matrix}\begin{matrix}{\overline{C}}_{eq} = \frac{\dot{M}}{2 π K x} \\ if K = x \cdot s\end{matrix} \\ where x is distance from the \\ source and radius of the NF \\ spherical prism .\end{matrix}$ | $\begin{matrix}{\overline{C}}_{eq} = \frac{\dot{M}}{\frac{1}{2} 6 ( {D}^{2} + D {h}_{bz} ) \times s} \\ where D is distance from the \\ source and {h}_{bz} is the height \\ of the prism , representing the \\ breathing zone .\end{matrix}$ |
| | | $\begin{matrix}if D = {h}_{bz} , the equation is \\ \begin{matrix}{\underline{C}}_{eq} = \frac{\dot{M}}{6 D \times D \times s} or \\ {\overline{C}}_{eq} = \frac{\dot{M}}{6 K D} \\ if K = D \cdot s\end{matrix}\end{matrix}$ |

As is illustrated in Table 3, in all three cases, concentration at equilibrium can be expressed in the form of an eddy diffusivity model. Assuming same values for K and distance from the pathogen source, all three expressions for equilibrium concentrations then provide satisfactorily similar results—even in the hexagonal prism representation given that 6 can be considered a satisfactory zero-order approximation to 2π, as 6 is within five percent of 2π.

The challenge in using an eddy diffusivity model is determining the appropriate value of K. It is noted that in the Gaussian solution, the K is assumed to be constant over the domain of spread of an aerosol containing a pathogen. The form of the equation for K in the second NF/FF two-box model (K=D·s) is similar to an existing expression K₀, where K₀=α·u·l, where a is a dimensionless parameter that can be determined experimentally; u is a representative velocity, and 1 is a representative length. By assuming that α=1, the existing expression can have the same form as:

K=D·s Eq. (10)

Some existing approaches provide eddy diffusivity that is independent on distance form the pathogen source. Such existing approaches yield an eddy diffusivity that depends on the mechanical ACH_{FF} and the overall volume V of a space in considerations (e.g., a volume of a room). Those existing approaches yield the following expressions for existing K₀ in meters² per second:

K₀=(0.52ACH_{FF}/3600+8.61×10⁻⁵)V^{2/3} Eq. (11)

and

K₀=V^{2/3}ACH_{FF}/3600. Eq. (12)

It is noted that CFD simulations over a wide of range of indoor parameters yield the following existing relationship for K₀ in meters² per second:

K₀=0.824V^{2/3}N^{-2/3}ACH_{FF}/3600 Eq. (13)

where N equals to the number of inlet vents for the room.

By combining the two representations of the eddy diffusivity in Eq. (10) and Eq. (12) and assuming that the product of D and s is a constant, an effective velocity s_{eff} (in meters per minute, for example) can be determined, where s_{eff} is consistent with a constant eddy diffusivity at all distances from the source: D·s_{eff}=K₀. The following expression can be obtained for s_{eff}:

$${s}_{eff} = \frac{{V}^{2 / 3} {ACH}_{FF} / 60}{D} \qquad Eq . ( 14 )$$

Similarly, expressions for s_{eff} can be written combining Eq. (10) with Eq. (11) and Eq. (13):

$$\begin{matrix}{s}_{eff} = \frac{( 0.52 \frac{{ACH}_{FF}}{3600} + 8.61 \times 1 {0}^{- 5} ) {V}^{\frac{2}{3}} \times 60}{D} & Eq . ( 15 ) \\ {s}_{eff} = \frac{0.8 \; 24 {V}^{2 / 3} {N}^{- 2 / 3} {ACH}_{FF} / 60}{D} & Eq . ( 16 )\end{matrix}$$

Because all three existing K₀ and ACH relationships are constant with respect to D, the product of D·s_{eff} is a constant, thus any change in s is inversely proportional to the change in D. Therefore, as D increases moving away from a source, the value of s_{eff} decreases.

Further, by leveraging existing relationships between α and u and l, a constant K in accordance with aspects of this disclosure can be defined as K=a·s_{eff}·D, where the parameter a can be defined as a means of capturing any dependency of K on distance from the source and adjustment to the dependence on ACH in the form (should measurement data indicate there are dependencies):

α=λD^{μ}·ηACH_{FF}^{γ} Eq. (17)

where λ, μ, η, and γ are power law coefficients should comparison of the model with real-world data suggest that adjustments are necessary.

Substituting the equation for K into Eq. (9) and rearranging so that the FSA of the hexagonal prism is calculated, ACH_{NF} can be expressed as:

$${ACH}_{NF} = \frac{\frac{6}{2} ( {D}^{2} + {Dh}_{b z} ) α \cdot {s}_{eff} \cdot 60}{{V}_{NF}} \qquad Eq . ( 18 )$$

To calculate an average exposure dose over a period of time, assuming that the initial concentration is zero (C(0)=0) from a single source, the average exposure dose can be estimated by determining the average concentration at the midpoint of the duration

$$( \frac{Δ t}{2} ) \text{:}$$

$${C}_{AVE} = \frac{\dot{M}}{{V}_{NF} \frac{{ACH}_{N F}}{60}} ( 1 - {e}^{- \frac{{ACH}_{NF} Δ t}{ 60 \; 2}} ) + \frac{\dot{M}}{{V}_{FF} \frac{{ACH}_{FF}}{60}} ( 1 - {e}^{- \frac{{ACH}_{FF} Δ t}{ 60 \; 2}} ) \qquad Eq . ( 19 )$$

As the duration increases, the factors

$$( 1 - {e}^{- \frac{{ACH}_{NF} Δ t}{ 60 \; 2}} ) and ( 1 - {e}^{- \frac{{ACH}_{FF} Δ t}{ 60 \; 2}} )$$

converge on 1. Given that ACH_{NF} can be greater than ACH_{FF}, the factor

$$( 1 - {e}^{- \frac{{ACH}_{NF} Δ t}{ 60 \; 2}} )$$

can converge faster than the factor

$$( 1 - {e}^{- \frac{{ACH}_{FF} Δ t}{ 60 \; 2}} ) .$$

That is, the near field term can achieve equilibrium faster than the far field term. It is noted that if the value of

$$\frac{{ACH}_{NF}}{ 60} \frac{Δ t}{2}$$

is large (e.g., greater than 3), there is a small difference between the concentrations at Δt or a period of time t.

### Multiple Sources

Equation (19) above provides an estimate of the average concentration from emission of one person at a receptor, but not the contribution of how emission from multiple persons can affect the average concentration. To address multiple sources in combination, the additivity property of the superposition principle of linear systems can be applied. The superposition principle permits determining the effect of each person's emissions at a receptor individually and then adding the individual effects in order to determine the collective effect of multiple sources.

Accordingly, if a NF and FF approach can be used to estimate the higher concentration in the proximity of one person to another person, then if N persons are added to the system, and n additional NF boxes also are added, the individual terms from each person (or emission source) of the N persons can be added to the equation defining the average concentration of pathogen. Each one of those individual terms being independent and collectively summing to total concentration.

$${C}_{AVE} = {\sum }_{P e = 1}^{N} {[ ( \frac{\dot{M}}{{V}_{NF} \frac{{ACH}_{NF}}{60}} ) ( 1 - {e}^{- \frac{{ACH}_{NF} Δ t}{ 60 \; 2}} ) + ( \frac{\dot{M}}{{V}_{FF} \frac{{ACH}_{F F}}{60}} ) ( 1 - {e}^{- \frac{{ACH}_{FF} Δ t}{ 60 \; 2}} ) ]}_{N} \qquad Eq . ( 20 )$$

The superposition principle also includes a homogeneity property that permits applying a scalar factor across all emission sources resulting in the concentration at the receptor changing proportionally to the value of the scalar. Accordingly, if the emission rate from each source is increased or decreased by a factor, then it can be assumed that the concentration would increase or decrease by the same factor. The scalar could also be the product of several scalars, including a probability factor. Embodiments of this disclosure can use the homogeneity property defining a scalar φ to adjust both the emission rate and the probability of the emission rate, assuming that the emission rate and the probability of emission rate are constant for all sources for a given scenario, resulting in the following equation:

$${C}_{AVE} = φ \dot{M} \times {\sum }_{So = 1}^{N} {[ ( \frac{1}{{V}_{NF} \frac{{ACH}_{NF}}{60}} ) ( 1 - {e}^{- \frac{{ACH}_{NF} Δ t}{ 60 \; 2}} ) + ( \frac{1}{{V}_{FF} \frac{{ACH}_{F F}}{60}} ) ( 1 - {e}^{- \frac{{ACH}_{FF} Δ t}{ 60 \; 2}} ) ]}_{N} \qquad Eq . ( 21 )$$

In a two-source system with one receptor, where the distance between each one of the two sources and the receptor is the same, Eq. (20) can be written as follows:

$${C}_{A V E} = φ \dot{M} \times {\sum }_{S o = 1}^{2} [ ( 1 - {e}^{- \frac{{ACH}_{NF} Δ t}{ 60 \; 2}} ) ( \frac{1}{{V}_{NF} \frac{{ACH}_{NF}}{60}} ) + ( 1 - {e}^{- \frac{{ACH}_{FF} Δ t}{ 60 \; 2}} ) ( \frac{1}{{V}_{FF} \frac{{ACH}_{FF}}{60}} ) ] \qquad Eq . ( 22 )$$

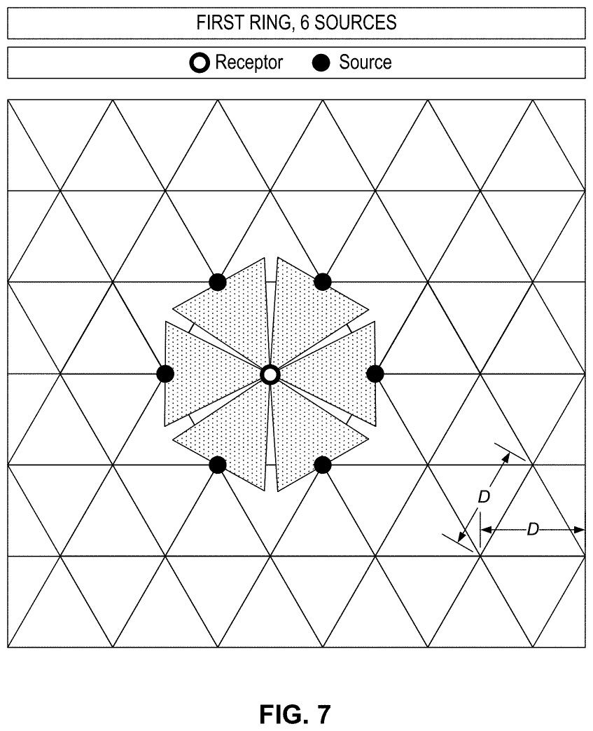

In order to evaluate Eq. (21), embodiments of this disclosure rely on a grid of hexagonal prisms to represent NF volumes. Using a hexagonal prism for the NF volume instead of a rectangular cuboid permits placing the system of equations on a regular grid of equidistant triangles (see FIG. 7). Using a regular grid of equilateral triangles, in contrast to a regular rectangular grid, has advantages because all nodes in the regular grid of equidistant triangles are equidistant from their nearest neighbors. This equidistant-neighbor aspect can be useful when assessing various distancing options. Further, each hexagonal prism contains two persons, one person at the center (e.g., a receptor) and another person at the outer vertex (e.g., a source). FIG. 7 shows near-field triangular prisms for the 1^{st} Ring of the group that have a height and base of D, in a triangular grid with each side length of D. The first ring's triangular prism's have a base area of A1=½ D². In FIG. 7, a source is represented by a solid circle, and a receptor is represented by an open circle.

The use of a triangular grid permits configuring an approximate hexagonal prism that is made up of six triangular prisms and approximates a cylinder. Each source can be centered on the six closest nodes to a receptor at the center of the hexagonal prism.

The regular grid of equilateral triangles, as is shown in FIG. 7, can be used to evaluate both the NF concentration and FF concentration for six sources at the receptor in the center, using the following equation:

$${C}_{AVE} = φ \dot{M} \times {\sum }_{So = 1}^{6} [ ( 1 - {e}^{- \frac{{ACH}_{NF - 1 st} Δ t}{ 60 \; 2}} ) ( \frac{1}{{V}_{NF} \frac{{ACH}_{NF - 1 st}}{60}} ) + ( 1 - {e}^{- \frac{{ACH}_{FF} Δ t}{ 60 \; 2}} ) ( \frac{1}{{V}_{FF} \frac{{ACH}_{FF}}{60}} ) ] \qquad Eq . ( 23 )$$

Referring back to Eq. (18), ACH_{NF} can be determined as follows:

$${ACH}_{NF - 1 st Ring} = \frac{\frac{6}{2} ( {D}^{2} + D {h}_{b z} ) α \cdot {s}_{eff} \cdot 60}{\frac{6}{2} {D}^{2} {h}_{b z}} \qquad Eq . ( 24 )$$

which can be simplified to yield

$${ACH}_{NF - 1 st Ring} = α \times {s}_{eff} \times ( \frac{1}{{h}_{b z}} + \frac{1}{D} ) \times 60 \qquad Eq . ( 25 )$$

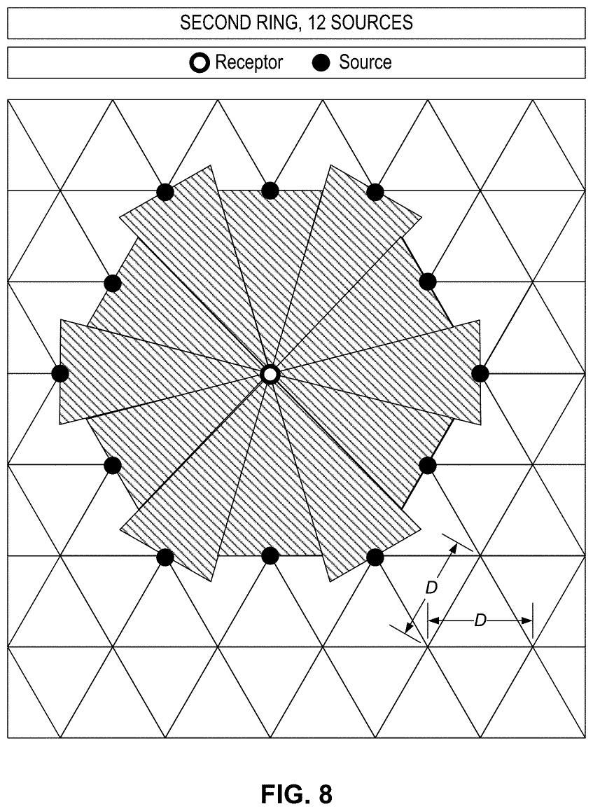

Similar to the six-source arrangement shown in FIG. 7, a 12-source arrangement can be configured as is shown in FIG. 8. The 12-source arrangement maintains each person (or source) within the arrangement at a D distance apart from one another. In that 12-source arrangement, each person (or source) is located at a distance that is approximately 2D from the receptor. In this case, instead of a hexagonal prism, a dodecagonal prism (12-sided prism) is drawn. In the 12-source arrangement, 1/12 of the emissions taken and 1/12 of the total dodecagonal prism volume is used. FIG. 8 shows NF triangular prisms for the 2^{nd} Ring of the group that is equally spaced by D. The second ring's triangular prisms have areas A2=½ 2D² and A2=½ \sqrt{3}D². These average to

$$A 2 = \frac{1}{2} {D}^{2} ( 1 + \frac{\sqrt{3}}{2} ) .$$

In FIG. 8, a source is represented by a solid circle, and a receptor is represented by an open circle.

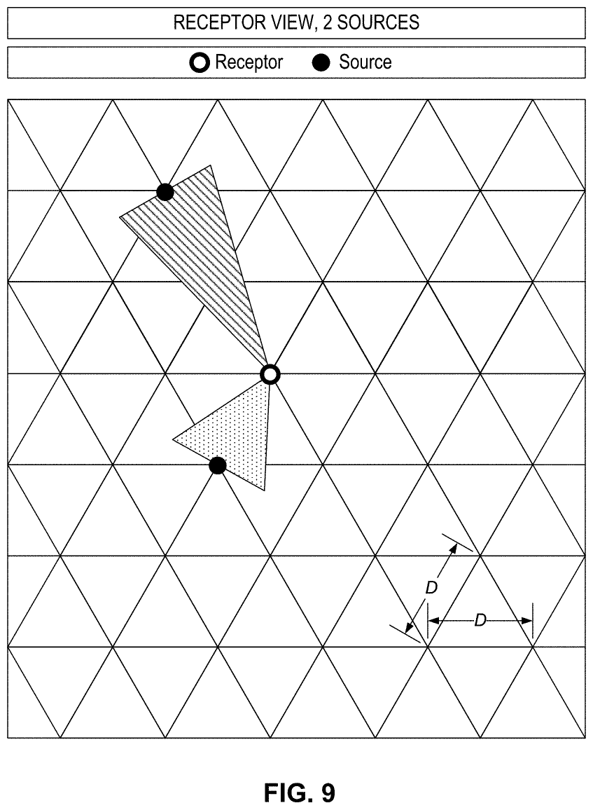

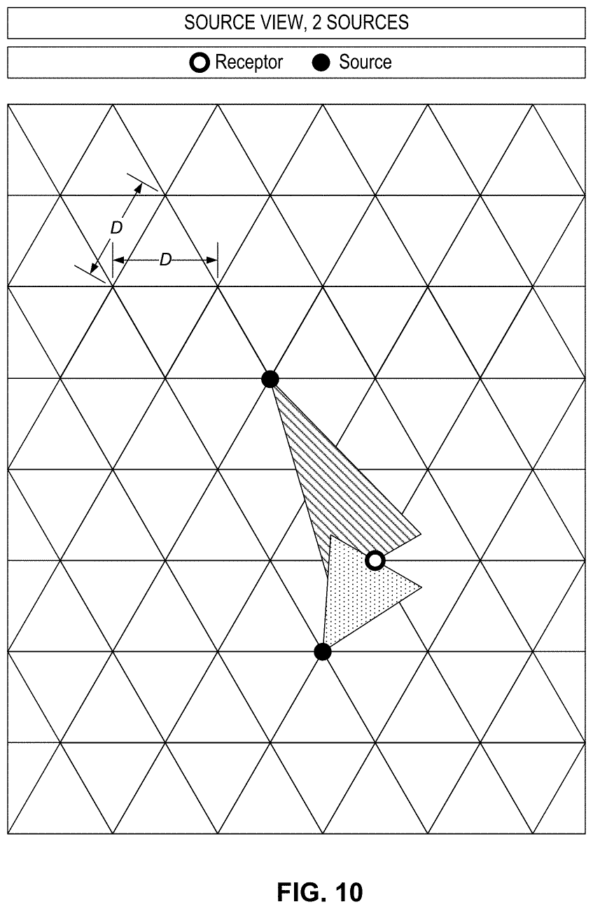

The orientation of the triangular prism within the box makes no difference to the determination of a concentration of pathogen. Accordingly, as is illustrated in FIG. 9, each of the triangular prisms can be rotated 180 degrees for visual convenience. As such, a configuration of sources and a receptor can be defined identically from two perspectives: the source view and the receptor view. FIG. 9 illustrates a receptor view. The same two triangles that impact the receptor in the source perspective can be turned 180 degrees and are part of the potential set of triangles in each ring. FIG. 10 shows a source view with two sources. Under an assumption of isotropy (assuming no predominate flow and a sufficient averaging period), the sources emit in all directions equally. It is noted that the dimensions and parameters are identical between both views. In FIG. 9 and FIG. 10, a source is represented by a solid circle and a source is represented by an open circle.

The ACH_{NF} can be determined using the dimension of the 1/12 triangular wedge which is derived as follows:

$${ACH}_{NF - 2 nd Ring} = \frac{\frac{1}{2} α \times {s}_{eff} \times ( 2 \times \frac{1}{2} {D}^{2} ( 1 + \frac{\sqrt{3}}{2} ) + {h}_{b z} \times D ) \times 60}{\frac{1}{2} {D}^{2} ( 1 + \frac{\sqrt{3}}{2} ) ( {h}_{b z} )} \qquad Eq . ( 26 )$$

which can be simplified to yield

$${ACH}_{NF - 2 nd Ring} = α \times {s}_{eff} \times ( \frac{1}{{h}_{b z}} + \frac{1}{( 1 + \frac{\sqrt{3}}{2} ) \times D} ) \times 60 \qquad Eq . ( 27 )$$

In some embodiments, any number of successive rings can be included in order to allow any number of persons greater than one. Each ring adds 6 additional people relative to the number of people present in the previous ring (e.g., the first ring holds 6 people, the second ring holds 12 people, the third ring holds 18, and so forth). Table 4 has the equations for the area of each of the triangular prisms, along with the equation used to calculate the ACH_{NF} for each ring.

TABLE 4
Triangular prism parameters and equations for each ring.

| | | | | | Triangles | Equation for the | Equation for Area | |
|---|---|---|---|---|---|---|---|---|
| | People | Cumulative | Triangles | Cumulative | per People | Area of Triangular | of Triangular Prism | ACH_{NF} Equation for |
| Ring # | per Ring | People | Per Ring | Triangles | by Ring | Prism | in Terms of A1 | each Ring |
| 1 | 6 | 6 | 6 | 6 | 1 | A1 = 0.5D² | A1 = A1 | α × s × (h_{bz} + D) × 60 |
| 2 | 12 | 18 | 18 | 24 | 3/2 | A2 = 0.933D² | A2 = A1 × 1.866 | α × s × (h_{bz} + 11.866 × D) × 60 |
| 3 | 18 | 36 | 30 | 54 | 5/3 | A3 = 1.40D² | A3 = A1 × 2.799 | α × s × (h_{bz} + 12.799 × D) × 60 |
| 4 | 24 | 60 | 42 | 96 | 7/4 | A4 = 1.87D² | A4 = A1 × 3.732 | α × s × (h_{bz} + 13.732 × D) × 60 |
| 5 | 30 | 90 | 54 | 150 | 9/5 | A5 = 2.33D² | A5 = A1 × 4.665 | α × s × (h_{bz} + 14.665 × D) × 60 |
| 6 | 36 | 126 | 66 | 216 | 11/6 | A6 = 2.80D² | A6 = A1 × 5.598 | α × s × (h_{bz} + 15.598 × D) × 60 |
| 7 | 42 | 168 | 78 | 294 | 13/7 | A7 = 3.27D² | A7 = A1 × 6.531 | α × s × (h_{bz} + 16.531 × D) × 60 |
| 8 | 48 | 216 | 90 | 384 | 15/8 | A8 = 3.73D² | A8 = A1 × 7.464 | α × s × (h_{bz} + 17.464 × D) × 60 |
| 9 | 54 | 270 | 102 | 486 | 17/9 | A9 = 4.20D² | A9 = A1 × 8.397 | α × s × (h_{bz} + 18.397 × D) × 60 |

Applying the superposition principle, the contribution of each person on the receptor at the center can be calculated. In an example scenario having 60 sources (four rings) C_{AVE} can be expressed as follows:

$${C}_{AVE} = φ \dot{M} \times ( {\sum }_{S o = 1}^{6} ( 1 - {e}^{- \frac{{ACH}_{NF - 1 s t} Δ t}{ 60 \; 2}} ) ( \frac{1}{{V}_{N F} \frac{{ACH}_{NF - 1 st}}{60}} ) + ( 1 - {e}^{- \frac{{ACH}_{FF} Δ t}{ 60 \; 2}} ) ( \frac{1}{{V}_{FF} \frac{{ACH}_{FF}}{60}} ) + {\sum }_{S o = 7}^{18} ( 1 - {e}^{- \frac{{ACH}_{NF - 2 n d} Δ t}{ 60 \; 2}} ) ( \frac{1}{{V}_{NF} \frac{{ACH}_{NF - 2 nd}}{60}} ) + ( 1 - {e}^{- \frac{{ACH}_{FF} Δ t }{ 60 \; 2}} ) ( \frac{1}{{V}_{FF} \frac{{ACH}_{FF}}{60}} ) + {\sum }_{S o = 19}^{36} ( 1 - {e}^{- \frac{{ACH}_{NF - 3 rd} Δ t}{ 60 \; 2}} ) ( \frac{1}{{V}_{N F} \frac{{ACH}_{NF - 3 rd}}{60}} ) + ( 1 - {e}^{- \frac{{ACH}_{FF} Δ t}{ 60 \; 2}} ) ( \frac{1}{{V}_{FF} \frac{{ACH}_{FF}}{60}} ) + {\sum }_{S o = 37}^{60} ( 1 - {e}^{- \frac{{ACH}_{NF - 4 t h} Δ t}{ 60 \; 2}} ) ( \frac{1}{{V}_{NF} \frac{A {C}^{1} {H}_{NF - 4 th}}{60}} ) + ( 1 - {e}^{- \frac{{ACH}_{FF} Δ t}{ 60 \; 2}} ) ( \frac{1}{{V}_{FF} \frac{{ACH}_{FF}}{60}} ) ) \qquad Eq . ( 28 )$$

The foregoing equation can be simplified by pulling out the factors common in the two terms and rearranging as follows:

$${C}_{AVE} = φ \dot{M} {So}_{Total} ( 1 - {e}^{- \frac{{ACH}_{FF} Δ t}{ 60 \; 2}} ) ( \frac{1}{{V}_{FF \frac{{ACH}_{FF}}{60}}} ) + \\ φ \dot{M} ( {\sum }_{So = 1}^{6} ( 1 - {e}^{- \frac{{ACH}_{NF - 1 s {t}^{Δ t}}}{ 60 \; 2}} ) ( \frac{1}{{V}_{NF \frac{{ACH}_{N F - 1 s t}}{60}}} ) + {\sum }_{So = 7}^{18} ( 1 - {e}^{- \frac{{ACH}_{NF - 2 n d} Δ t}{ 60 \; 2}} ) ( \frac{1}{{V}_{NF \frac{{ACH}_{NF - 2 nd}}{60}}} ) + {\sum }_{So = 19}^{36} ( 1 - {e}^{- \frac{{ACH}_{NF - 3 {rd}^{Δ t}}}{ 60 \; 2}} ) ( \frac{1}{{V}_{NF \frac{{ACH}_{NF - 3 rd}}{60}}} ) + {\sum }_{So = 37}^{60} ( 1 - {e}^{- \frac{{ACH}_{NF - 4 th} Δ t}{ 60 \; 2}} ) ( \frac{1}{{V}_{NF \frac{{ACH}_{NF - 4 th}}{60}}} ) ) \qquad Eq . ( 29 )$$

Calculating ACH_{FF} and ACH_{NF}

For purposes of determining the far-field concentration term, ACH_{FF} should include any mechanisms that remove air from the space (e.g., natural ventilation, infiltration, mechanical ventilation, a combination thereof, or similar), mechanisms that remove the contaminant from the space (e.g., filtration and deposition), and mechanisms that inactivate contaminants (e.g., reaction, temperature, humidity, radiation, a combination thereof, or similar). Accordingly,

ACH_{FF}=ACH_{Nat.l Vent}+ACH_{Infiltr.}+ACH_{Mech.Vent}+ACH_{HVAC Re.}+ACH_{Inact.}+ACH_{Dep} Eq. (30)

The ACH_{HVAC Recirc.} can be based upon the flow rate and the portion of the recirculated air from which any contaminants has been removed (ACH_{HVAC Re}×Ef_{Filter}):

ACH_{FF}=ACH_{Nat.l Vent}+ACH_{Infiltr.}+ACH_{Mech.Vent}+ACH_{HVAC Re.}×Ef_{Filter}+ACH_{Inact.}+ACH_{Dep} Eq. (31)

For purposes of using the ACH_{FF} to determine the ACH_{NF} using Eq. (18) by first determining the s_{eff}, the ACH_{FF} should include mechanisms that result in actual air flow. Thus, the ACH_{Inact.} and ACH_{Dep} have not been included and the unreduced ACH_{HVAC Recirc} should be used, resulting in the following:

ACH_{FF}_{_{Eddy Diff}}=ACH_{Nat.l Vent}+ACH_{Infiltr.}+ACH_{Mech.Vent}+ACH_{HVAC Re.} Eq. (32)

For the final ACH_{NF}, the ACH_{Inact.} and ACH_{Dep} should be added back in, as shown below for the 1st ring of sources:

$${ACH}_{NF - 1 st Ring} = α \times {s}_{eff} \times ( \frac{1}{{h}_{b z}} + \frac{1}{D} ) \times 60 + {ACH}_{Inact .} + {ACH}_{Dep} \qquad Eq . ( 33 )$$

### Exposure Model

An inhalation exposure dose model in accordance with this disclosure uses C_{AVE} to determine an average mass of inhaled contaminant:

Eₘₐₛₛ=C_{AVE}×Qᵢₙₕₐₗₐₜᵢₒₙ×Δt Eq. (34)

where Eₘₐₛₛ is the mass of inhaled contaminant (mass), C_{AVE} is the average air concentration (mass/m³) over the duration of exposure, Qᵢₙₕₐₗₐₜᵢₒₙ is the inhalation rate (m³/min), and Δt is the duration of exposure (min).

As mentioned in connection with Eq. (1), because this model can be viewed from a worker safety perspective, a total exposure dose of all people in an activity space can be obtained by multiplying the total number of people (denoted by Pe_{Exposed}), an assuming average contaminant concentration and the same duration Δt in the activity space (e.g., region 110). As a result,

Eₘₐₛₛ=C_{AVE}×Qᵢₙₕₐₗₐₜᵢₒₙ×Δt×Pe_{Exposed} Eq. (35)

As is described herein, concentration contributions can be determined for a person assumed to be at the center of a triangular grid where people are spaced equidistantly (based upon the distancing specified by data from a source device, for example). Contaminant concentration at the center is assumed to be representative (albeit conservative) for all people in the group since (1) location of each person is unlikely to be static during the activity and (2) exposure is driven primarily by close-in sources (e.g., other people) and all persons in the group have close-in sources.

In scenarios where mask effectiveness is included in the exposure model, recognizing that there is an effect on both the inhalation side (1−Efᵢₙ) and on the exhalation side (1−Efₒᵤₜ), the foregoing equation takes the following form:

Eₘₐₛₛ=(1−Efₒᵤₜ)×C_{AVE}×Qᵢₙₕₐₗₐₜᵢₒₙ×(1−Efᵢₙ)×Δt×Pe_{Exposed} Eq. (36)

Equation (35) determines the total exposure dose that a worst-case person (located at a receptor at the center of all rings accommodating Pe_{Exposed} persons) can receive if all people in the group emit at a rate \dot{M} for the exposure duration. It is noted that the exposure model that yields Eq. (36) assumes that all people are emitters (that is, all people are assumed to be infected), when in fact only a few may be emitters. Based upon the homogeneity property of the superposition principle, φ, in the expanded dose equation can be the likelihood that a person is infected, as is shown below.

$${E}_{mass} = ( 1 - E {f}_{out} ) \times {Q}_{inhalation} \times ( 1 - E {f}_{in} ) \times Δt \times {Pe}_{E xposed} \times ( φ \dot{M} {Pe}_{Emitting} ( 1 - {e}^{- \frac{{ACH}_{FF} Δ t}{ 60 \; 2}} ) ( \frac{1}{{V}_{FF} \frac{{ACH}_{FF}}{60}} ) + \\ φ \dot{M} ( {\sum }_{Pe = 1}^{6} ( 1 - {e}^{- \frac{{ACH}_{NF - 1 s {t}^{Δ t}}}{ 60 \; 2}} ) ( \frac{1}{{V}_{NF} \frac{{ACH}_{N F - 1 s t}}{60}} ) + {\sum }_{Pe = 7}^{18} ( 1 - {e}^{- \frac{{ACH}_{NF - 2 n d} Δ t}{ 60 \; 2}} ) ( \frac{1}{{V}_{NF} \frac{{ACH}_{NF - 2 nd}}{60}} ) + {\sum }_{Pe = 19}^{36} ( 1 - {e}^{- \frac{{ACH}_{NF - 3 {rd}^{Δ t}}}{ 60 \; 2}} ) ( \frac{1}{{V}_{NF} \frac{{ACH}_{NF - 3 rd}}{60}} ) + {\sum }_{Pe = 37}^{60} ( 1 - {e}^{- \frac{{ACH}_{NF - 4 th} Δ t}{ 60 \; 2}} ) ( \frac{1}{{V}_{NF} \frac{{ACH}_{NF - 4 th}}{60}} ) … ) \qquad Eq . ( 37 )$$

It is noted that Eq. (36) contemplates at least 60 individual in the group of people for which exposure risk is being determined. In cases where there are less than 60 people, the series over rings in Eq. (36) are adjusted according to the number of populated rings

Equation (36) can be expressed more succinctly, as follows:

Eₘₐₛₛ=(1−Efₒᵤₜ)×(1−Efᵢₙ)×Δt×Pe_{Exposed}×φ\dot{M}×(Pe_{Emitting}FF_{Factor}+NF_{Factor}) Eq. (38)

where the following expressions have been introduced:

$$\begin{matrix} {Pe}_{Emitting} = P {e}_{Exposed} - 1 & Eq . ( 39 ) \\ {FF}_{Factor} = ( 1 - {e}^{- \frac{{ACH}_{FF} Δ t}{ 60 \; 2}} ) ( \frac{1}{{V}_{FF} \frac{{ACH}_{FF}}{60}} ) & Eq . ( 40 ) \\ {NF}_{Factor} = {\sum }_{Pe = 1}^{6} ( 1 - {e}^{- \frac{{ACH}_{NF - 1 s {t}^{Δ t}}}{ 60 \; 2}} ) ( \frac{1}{{V}_{NF} \frac{{ACH}_{N F - 1 s t}}{60}} ) + {\sum }_{Pe = 7}^{18} ( 1 - {e}^{- \frac{{ACH}_{NF - 2 n d} Δ t}{ 60 \; 2}} ) ( \frac{1}{{V}_{NF} \frac{{ACH}_{NF - 2 nd}}{60}} ) + {\sum }_{Pe = 19}^{36} ( 1 - {e}^{- \frac{{ACH}_{NF - 3 {rd}^{Δ t}}}{ 60 \; 2}} ) ( \frac{1}{{V}_{NF} \frac{{ACH}_{NF - 3 rd}}{60}} ) + {\sum }_{Pe = 37}^{60} ( 1 - {e}^{- \frac{{ACH}_{NF - 4 th} Δ t}{ 60 \; 2}} ) ( \frac{1}{{V}_{NF} \frac{{ACH}_{NF - 4 th}}{60}} ) … ) & Eq . ( 41 )\end{matrix}$$

As mentioned, in cases where there are less than 60 people, NF_{Factor} is adjusted according to the number of populated rings.

As is described herein, the exposure risk scoring model of this disclosure can take the form of a relative inhalation dose model that compares a specific evaluated scenario to a defined high-risk BL scenario by using a ratio of (a) an average contaminant concentration for a duration Δt, in an activity space, for the evaluated scenario and (b) the average contaminant concentration for a duration Δt, in the activity space, for the defined high-risk BL scenario. Accordingly, the risk score Σ can be defined as follows.

$$ɛ = \frac{{E}_{mass i}}{{E}_{mass BL}} = \frac{{φ}_{i}}{{φ}_{B L}} \times \frac{{\dot{M}}_{i}}{{\dot{M}}_{B L}} \times \frac{{( P {e}_{Emitting} {FF}_{Factor} + {NF}_{Factor} )}_{i}}{{( {Pe}_{Emittin g} {FF}_{Factor} + {NF}_{Factor} )}_{B L}} \times \frac{{( 1 - E {f}_{o u t} )}_{i}}{{( 1 - E {f}_{o u t} )}_{B L}} \times \frac{{( 1 - E {f}_{i n} )}_{i}}{{( 1 - E {f}_{i n} )}_{B L}} \times \frac{{Q}_{{inhalation}_{i}}}{{Q}_{{inhalation}_{B L}}} \times \frac{P {e}_{E {xposed}_{i}}}{P {e}_{E {xposed}_{B L}}} \times \frac{Δ {t}_{i}}{Δ {t}_{B L}} \times \frac{Immunity {Factor}_{i}}{Immunity {Factor}_{B L}} \times \frac{Variant {Factor}_{i}}{Variant {Factor}_{B L}} \times \frac{Test {Factor}_{i}}{Test {Factor}_{B L}} \qquad Eq . ( 42 )$$

Without intending to be bound by modeling, the consideration of the likelihood that any individual member of the group is infectious at the start of the scenario or modeled event, with the likelihood of infection represented by the variable cp. In this disclosure, the range of likelihood of infectiousness in the group can range from 1 (certain infectiousness) on the high end, to a value on the low end that is 100 times less than what is estimated as the community average infectiousness. In scenarios contemplated in this disclosure, it is assumed that at least one person is not infectious, so the population that could be infectious is the size of the group, Pe, minus 1.

Without intending to be bound by modeling, the risk assessment subsystem 250 (FIG. 2) can include the estimate the community average infectiousness by using the reported 7-day average per 100,000 of diagnosed cases, denoted by Cases_{Per 100000}, an estimate of the ratio of the undiagnosed cases over the diagnosed cases (R_{Undiag}), and the average length of an infectiousness in days, (D_{Inf}), multiplied by the subgroup factor, which is the adjustment of the subgroup's rate of infectiousness as compared to the rate of infectiousness of the community.

$$φ = ( 1 - {( 1 - {R}_{Undiag} \times \frac{{Cases}_{Per 100000}}{100 \text{,} 000} )}^{{D}_{Inf}} ) 100 \text{,} 000 \times Subgroup Factor \qquad Eq . ( 43 )$$

Within a community, the population can be subdivided into subgroups or as follows:

(I) Subgroup Factor=0.01. The group is composed of people who, prior to the event are estimated as having a likelihood infection that is 100 times lower than the community's average due to their adhering to public health guidance on distancing, masking, and exposure to crowds/people.

(II) Subgroup Factor=0.1. The group is composed of people who, prior to the event are estimated as having a likelihood infection that is 10 times lower than the community's average due to their adhering to public health guidance on distancing, masking, and exposure to crowds/people.

(III) Subgroup Factor=1. The group is composed of people who, prior to the event are estimated as having a likelihood infection that is equal to the community's average.

(IV) Subgroup Factor=0.1. The group is composed of people who, prior to the event are estimated as having a likelihood infection that is 10 times higher than the community's average due to their not adhering to public health guidance on distancing, masking, and exposure to crowds/people.

(V)φ=1. The group is comprised of people who are known to be infectious.

It is noted that the foregoing subgroup factors also can be assigned to individual persons instead of a subgroup. In other words, the subgroup factors also can be applied to a subsgroup consisting of a single person.

In connection with variant prevalence, again without intending to be bound by modeling, embodiments of this disclosure can adjust the equivalent exposure dose upward based on increased transmission rates of some variants. Thus, as an example, B.1.1.7 is 50% more transmissive and, thus, a dose of B.1.1.7 variant is more potent. Accordingly, embodiments of this disclosure can use a higher equivalent exposure value. Such a value for a particular variant can be configured, in some cases, according to published greater transmissivity of that particular variant relative to the wild type.

Embodiments of this disclosure can incorporate immunities—both immunity stemming from vaccination and immunity stemming from recovery from infection—in two ways: (I) A reduction in the rate of virus shedding of immunized persons who do become infected, thus reducing M for the fraction of people with immunity. (II) As a barrier to infection with an effectiveness that, in some embodiments, can be equal to known efficacy based conceptually on the model used by the EPA for dose and exposure definition. Embodiments of this disclosure treat immunity gained by recovery from infection is equal to immunity gained from vaccination.

Embodiments of this disclosure can incorporate efficacy and timing of testing regimes relative to the days an individual may be expected to be infectious. Without intending to be bound by modeling, it can be assumed that if an individual is infectious at the time of an event—e.g., at an instant when risk assessment subsystem 250 determines a risk factor using the concentration model of this disclosure—the timing of the infection prior to the event is a uniform distribution. For example, if a number of days since infection, denoted by N_{D}, is equal to five, and testing was conducted three days prior to the event, there is a ⅗ probability that they were infected when they were tested and a ⅖ probability they got infected after they were tested (in the two subsequent days before the event). Assuming a testing FN rate (T_{FN}) of 10% and that testing was performed three days before the event, the testing adjustment factor can be determined as follows.

In situations in which N_{D} is less than three, there is no adjustment to the likelihood that an individual is infectious because testing was performed prior to anyone becoming infectious. In situations in which N_{D} is three or more, a testing adjustment factor α_{T} can be determined as the weighted likelihood of either (a) having been infected at the time of testing and obtaining a false negative test or (b) becoming infected after the test. That is, α_{T}=(N_{D}−2)/N_{D}*T_{FN}+2/N_{D}. In some cases, as mentioned, T_{FN} can be equal to 0.10.

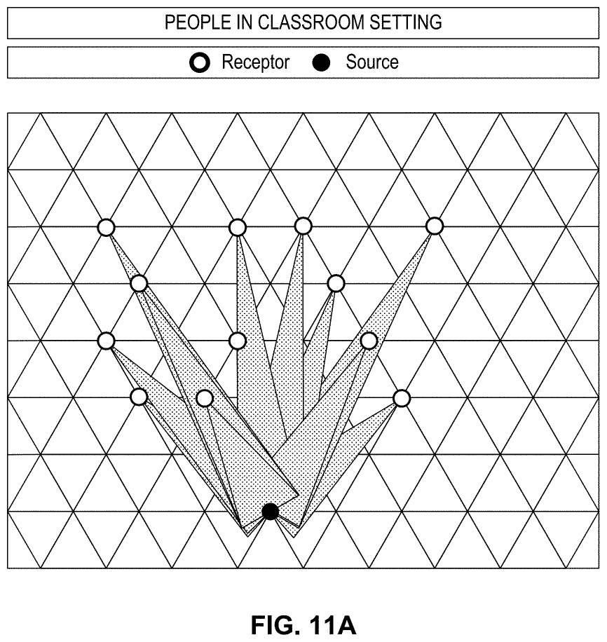

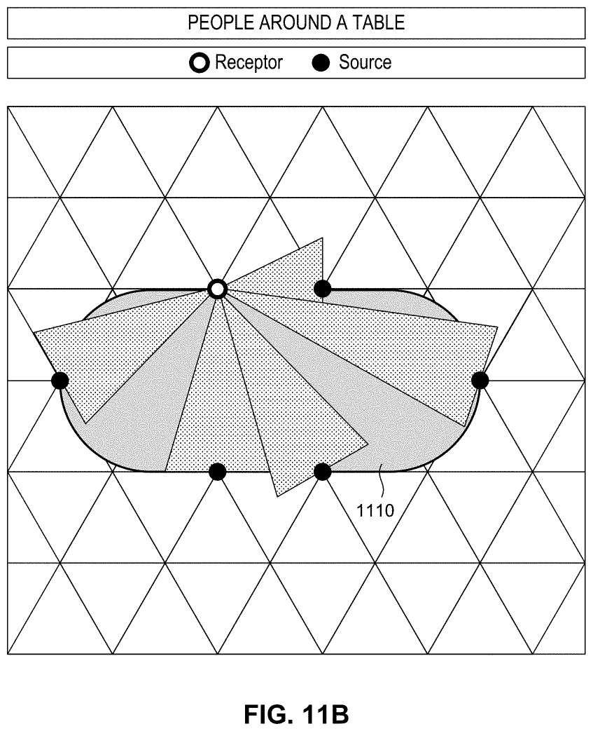

Embodiments of this disclosure can be applied to numerous exposure scenarios. For example, the risk assessment subsystem 250 (FIG. 2) can generate risk scores for exposure scenarios receptors at different positions in a cluster of people or exposure scenarios having concentrations of people located in a specific location in an activity space. For purposes of illustration, FIG. 11A illustrates triangular prisms NF and FF for an exposure scenario including people in a classroom setting. In that scenario, sources are represented with solid circles and a receptor is represented with an open circle. Further FIG. 11B illustrates triangular prisms NF and FF for an exposure scenario including people around a table 1110. Again, in that scenario, sources are represented with solid circles and a receptor is represented with an open circle. The resolution of the triangular grid can be adjusted so that many configuration can be modeled.

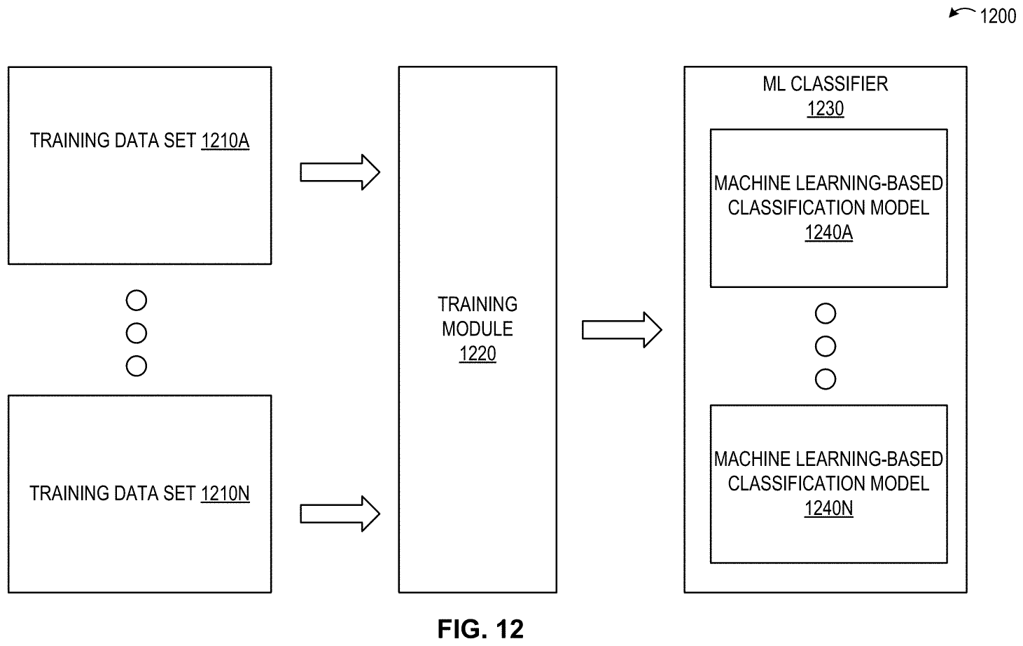

FIG. 12 illustrates an example system 200 for determining exposure to airborne pathogens. The system 200 may use machine learning techniques to train, based on an analysis of one or more training datasets 1210A-1210N by a training module 1220, at least one machine learning-based classifier 1230 (of the machine learning model configured with the computing device 106, etc.) that is configured to classify features extracted from one or more images, such as features relating to persons (e.g, a number of persons in an area, dimensions of an area, objects worn by individuals in an area (e.g, masks, clothing, personal protective equipment, etc.), and/or the like. The machine learning-based classifier 1230 may classify features extracted from one or more images to determine exposure to airborne pathogens.

The one or more training datasets 1210A-1210N may comprise labeled baseline data such as labeled data relating to persons (e.g, a number of persons in an area, dimensions of an area, objects worn by individuals in an area (e.g, masks, clothing, personal protective equipment, etc.), and/or the like. The labeled baseline data may include any number of feature sets.

The labeled baseline data may be stored in one or more databases. Data determined/extracted from a content item may be randomly assigned to a training dataset or a testing dataset. The assignment of data to a training dataset or a testing dataset may not be completely random. In this case, one or more criteria may be used during the assignment, such as ensuring that similar features relating to persons (e.g, a number of persons in an area, dimensions of an area, objects worn by individuals in an area (e.g, masks, clothing, personal protective equipment, etc.), and/or the like, dissimilar modal features relating to persons (e.g, a number of persons in an area, dimensions of an area, objects worn by individuals in an area (e.g, masks, clothing, personal protective equipment, etc.), and/or the like, and/or the like may be used in each of the training and testing datasets. In general, any suitable method may be used to assign the data to the training or testing datasets.

The training module 1220 may train the machine learning-based classifier 1230 by extracting a feature set from the labeled baseline data according to one or more feature selection techniques. In some instances, the training module 1220 may further define the feature set obtained from the labeled baseline data by applying one or more feature selection techniques to the labeled baseline data in the one or more training datasets 1210A-1210N. The training module 1220 may extract a feature set from the training datasets 1210A-1210N in a variety of ways. The training module 1220 may perform feature extraction multiple times, each time using a different feature-extraction technique. In some instances, the feature sets generated using the different techniques may each be used to generate different machine learning-based classification models 1240. In an embodiment, the feature set with the highest quality metrics may be selected for use in training. The training module 1220 may use the feature set(s) to build one or more machine learning-based classification models 1240A-1240N that are configured to determine exposure to airborne pathogens.

The training datasets 1210A-1210N and/or the labeled baseline data may be analyzed to determine any dependencies, associations, and/or correlations exposure factors (such as factors identified in and/or relating to Tables 1-3, etc.) in the training datasets 1210A-1210N and/or the labeled baseline data. The term “feature,” as used herein, may refer to any characteristic of an item of data that may be used to determine whether the item of data falls within one or more specific categories. By way of example, the features described herein may comprise any data/information relating to persons (e.g, a number of persons in an area, dimensions of an area, objects worn by individuals in an area (e.g, masks, clothing, personal protective equipment, etc.), exposure risk, pathogen analysis, and/or the like.

A feature selection technique may comprise one or more feature selection rules. The one or more feature selection rules may comprise determining which features in the labeled baseline data appear over a threshold number of times in the labeled baseline data and identifying those features that satisfy the threshold as candidate features. For example, any feature that appears greater than or equal to 2 times in the labeled baseline data may be considered as candidate features. Any features appearing less than 2 times may be excluded from consideration as a feature. A single feature selection rule may be applied to select features or multiple feature selection rules may be applied to select features. The feature selection rules may be applied in a cascading fashion, with the feature selection rules being applied in a specific order and applied to the results of the previous rule. For example, the feature selection rule may be applied to the labeled baseline data to determine exposure to airborne pathogens. A final list of candidate features may be analyzed according to additional features.

Determining exposure to airborne pathogens may be based on a wrapper method. A wrapper method may be configured to use a subset of features and train the machine learning model using the subset of features. Based on the inferences that are drawn from a previous model, features may be added and/or deleted from the subset. Wrapper methods include, for example, forward feature selection, backward feature elimination, recursive feature elimination, combinations thereof, and the like. In some instances, forward feature selection may be used to identify one or more candidate features relating to persons (e.g., a number of persons in an area, dimensions of an area, objects worn by individuals in an area (e.g., masks, clothing, personal protective equipment, etc.), exposure factors, and/or the like. Forward feature selection is an iterative method that begins with no feature in the machine learning model. In each iteration, the feature which best improves the model is added until an addition of a new variable does not improve the performance of the machine learning model. In an embodiment, backward elimination may be used to identify one or more candidate features relating to persons (e.g., a number of persons in an area, dimensions of an area, objects worn by individuals in an area (e.g., masks, clothing, personal protective equipment, etc.), exposure factors, and/or the like. Backward elimination is an iterative method that begins with all features in the machine learning model. In each iteration, the least significant feature is removed until no improvement is observed on removal of features.

Recursive feature elimination may be used to identify one or more candidate features relating to persons (e.g., a number of persons in an area, dimensions of an area, objects worn by individuals in an area (e.g., masks, clothing, personal protective equipment, etc.), exposure factors, and/or the like. Recursive feature elimination is a greedy optimization algorithm which aims to find the best performing feature subset. Recursive feature elimination repeatedly creates models and keeps aside the best or the worst performing feature at each iteration. Recursive feature elimination constructs the next model with the features remaining until all the features are exhausted. Recursive feature elimination then ranks the features based on the order of their elimination.

Exposure risk to pathogens may be determined according to an embedded method. Embedded methods include, for example, Least Absolute Shrinkage and Selection Operator (LASSO) and ridge regression which implement penalization functions to reduce overfitting. For example, LASSO regression performs L1 regularization which adds a penalty equivalent to the absolute value of the magnitude of coefficients and ridge regression performs L2 regularization which adds a penalty equivalent to the square of the magnitude of coefficients.

In some embodiments, probability distributions can be generated for each feature set that the training model has generated. Co-dependencies among such features also can be determined. Subsequently, Monte Carlo simulations can be performed to generate a distribution of potential relative risk values in order to quantify uncertainty in the exposure estimates, for a group od persons or an individual.

Further, after training module 1220 has generated a feature set(s), the training module 1220 may generate a machine learning-based predictive model 1240 based on the feature set(s). In some embodiments, the machine learning-based predicted model 1240 can be included into the models 254 (FIG. 2) to be applied by the risk assessment subsystem 250 (FIG. 2), for example. The machine learning-based predictive model 1240 can predict characteristics of a group of persons. Those characteristics can the be utilized in the exposure model to determine concentration of the group of persons or an individual within the group of persons. Machine learning-based predictive model, may refer to a complex mathematical model for data classification that is generated using machine-learning techniques. In one example, this machine learning-based classifier may include a map of support vectors that represent boundary features. By way of example, boundary features may be selected from, and/or represent the highest-ranked features in, a feature set.

In an embodiment, the training module 1220 may use the feature sets extracted from the training datasets 1210A-1210N and/or the labeled baseline data to build a machine learning-based classification model 1210A-1210N to determine exposure (and/or a risk of exposure) to airborne pathogens. In some examples, the machine learning-based classification models 1240A-1240N may be combined into a single machine learning-based classification model 1240. Similarly, the machine learning-based classifier 1230 may represent a single classifier containing a single or a plurality of machine learning-based classification models 1240 and/or multiple classifiers containing a single or a plurality of machine learning-based classification models 1240. The machine learning-based classifier 1230 may also include each of the training datasets 1210A-1210N and/or each feature set extracted from the training datasets 1210A-1210N and/or extracted from the labeled baseline data.

The extracted features relating to persons (e.g., a number of persons in an area, dimensions of an area, objects worn by individuals in an area (e.g., masks, clothing, PPE, etc.), exposure factors, and/or the like may be combined in a classification model trained using a machine learning approach such as discriminant analysis; decision tree; a nearest neighbor (NN) algorithm (e.g., k-NN models, replicator NN models, etc.); statistical algorithm (e.g., Bayesian networks, etc.); clustering algorithm (e.g., k-means, mean-shift, etc.); neural networks (e.g., reservoir networks, artificial neural networks, etc.); support vector machines (SVMs); logistic regression algorithms; linear regression algorithms; Markov models or chains; principal component analysis (PCA) (e.g., for linear models); multi-layer perceptron (MLP) ANNs (e.g., for non-linear models); replicating reservoir networks (e.g., for non-linear models, typically for time series); random forest classification; a combination thereof and/or the like. The resulting machine learning-based classifier 1230 may comprise a decision rule or a mapping that uses data/information relating to persons (e.g., a number of persons in an area, dimensions of an area, objects worn by individuals in an area (e.g., masks, clothing, personal protective equipment, etc.), exposure factors, and/or the like, such as image data, etc. to determine exposure to airborne pathogens.

The modal content information and the machine learning-based classifier 1230 may be used to predict emotions (and/or the like) for the test samples in the test dataset. In one example, the result for each test sample includes a confidence level that corresponds to a likelihood or a probability that the corresponding test sample accurately determines the exposure and/or risk of exposure to airborne pathogens. The confidence level may be a value between zero and one that represents a likelihood that the exposure and/or risk of exposure to airborne pathogens consists with a computed value. Multiple confidence levels may be provided for each test sample and each candidate prediction of exposure to an airborne pathogen. A top-performing candidate prediction of exposure to an airborne pathogen may be determined by comparing the result obtained for each test sample with a computed prediction of exposure to an airborne pathogen for each test sample. In general, the top-performing candidate prediction of exposure to an airborne pathogen will have results that closely match the computed prediction of exposure to an airborne pathogen. The top-performing candidate prediction of exposure to an airborne pathogen may be used to determine exposure to airborne pathogens.

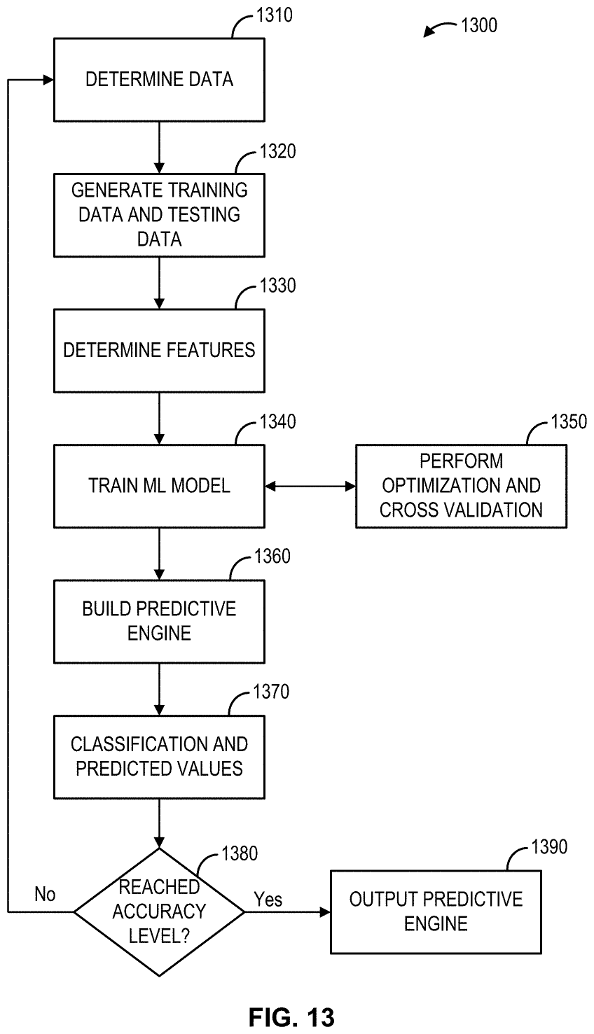

FIG. 13 is a flowchart illustrating an example training method 1300 for generating the machine learning classifier 1230 using the training module 1220. The training module 1220 can implement supervised, unsupervised, and/or semi-supervised (e.g., reinforcement-based) machine learning-based classification models 1240. Training method 1300, shown in FIG. 13, is an example of a supervised learning method; variations of this example of training method are discussed below, however, other training methods can be analogously implemented to train unsupervised and/or semi-supervised machine learning (predictive) models.

The training method 1300 may determine information determining exposure to airborne pathogens at 1310. The information for determining exposure to airborne pathogens may contain one or more datasets. Each dataset may include labeled baseline data.

The training method 1300 may generate, at 1320, a training dataset and a testing dataset. The training dataset and the testing dataset may be generated by calculating and/or computing an exposure to an airborne pathogen based on historical determinations of exposure to airborne pathogens. The training dataset and the testing dataset may be generated by randomly assigning data (for determining airborne pathogens) to either the training dataset or the testing dataset. In some instances, the assignment of data as training or test samples may not be completely random. In some instances, only the labeled baseline data for a specific feature extracted from data associated with determining exposure to pathogens, such as image data, etc., may be used to generate the training dataset and the testing dataset. In some instances, a majority of the labeled baseline data extracted from data associated with determining exposure to pathogens, such as image data, etc. may be used to generate the training dataset. For example, 75% of the labeled baseline data for predicting exposure to an airborne pathogen extracted from the data may be used to generate the training dataset and 25% may be used to generate the testing dataset. Any method or technique may be used to create the training and testing datasets.

The training method 1300 may determine (e.g., extract, select, etc.), at 1330, one or more features that can be used by, for example, a classifier to label features relating to persons (e.g., a number of persons in an area, dimensions of an area, objects worn by individuals in an area (e.g., masks, clothing, personal protective equipment, etc.), exposure factors, and/or the like. The training method 1300 may determine a set of training baseline features from the training dataset.

The training method 1300 may train one or more machine learning models using the one or more features at 1340. In some instances, the machine learning models may be trained using supervised learning. In another embodiment, other machine learning techniques may be employed, including unsupervised learning and semi-supervised. The machine learning models trained at 1340 may be selected based on different criteria and/or data available in the training dataset. For example, machine learning classifiers can suffer from different degrees of bias. Accordingly, more than one machine learning model can be trained at 1340, optimized, improved, and cross-validated at 1350.

The training method 1300 may select one or more machine learning models to build a predictive model at 1360 (e.g., a machine learning classifier, a predictive model, etc.). The predictive engine may be evaluated using the testing dataset. The predictive engine may analyze the testing dataset and generate classification values and/or predicted values at 1370. Classification and/or prediction values may be evaluated at 1380 to determine whether such values have achieved a desired accuracy level. Performance of the predictive engine may be evaluated in a number of ways based on a number of true positives, false positives, true negatives, and/or false negatives classifications of the plurality of data points indicated by the predictive engine. For example, the false positives of the predictive engine may refer to a number of times the predictive engine incorrectly determined exposure and/or a risk of exposure to an airborne pathogen and/or the like. Conversely, the false negatives of the predictive engine may refer to a number of times the machine learning model determined exposure and/or a risk of exposure to an airborne pathogen and/or the like incorrectly, when in fact, the determined exposure and/or a risk of exposure to an airborne pathogen and/or the like matches an actual exposure and/or risk of exposure. True negatives and true positives may refer to a number of times the predictive engine correctly determined exposure and/or a risk of exposure to an airborne pathogen and/or the like. Related to these measurements are the concepts of recall and precision. Generally, recall refers to a ratio of true positives to a sum of true positives and false negatives, which quantifies a sensitivity of the predictive engine. Similarly, precision refers to a ratio of true positives a sum of true and false positives.

When such a desired accuracy level is reached, the training phase ends and the predictive engine may be output at 1390; when the desired accuracy level is not reached, however, then a subsequent iteration of the training method 1300 may be performed starting at 310 with variations such as, for example, considering a larger collection of data relating to exposure to airborne pathogens.

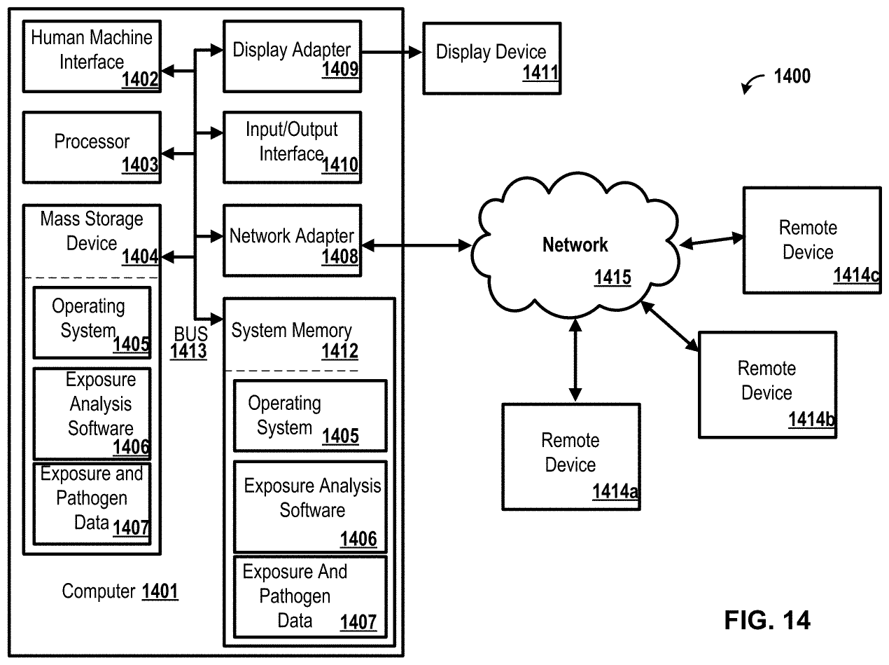

FIG. 14 shows an example system 1400 for determining exposure to airborne pathogens. In some embodiments, a device and/or module described herein (e.g., the user device 102, the computing device 106, etc.) may be embodied in a computer 1401 as is shown in FIG. 14.

The computer 1401 may comprise one or more processors 1403, a system memory 1412, and a bus 1413 that couples various components of the computer 1401 including the one or more processors 1403 to the system memory 1412. In the case of multiple processors 1403, the computer 1401 may utilize parallel computing.

The bus 1413 may comprise one or more of several possible types of bus structures, such as a memory bus, memory controller, a peripheral bus, an accelerated graphics port, and a processor or local bus using any of a variety of bus architectures.

The computer 1401 may operate on and/or comprise a variety of computer-readable media (e.g., non-transitory). Computer-readable media may be any available media that is accessible by the computer 1401 and comprises, non-transitory, volatile and/or non-volatile media, removable and non-removable media. The system memory 1412 has computer-readable media in the form of volatile memory, such as random access memory (RAM), and/or non-volatile memory, such as read-only memory (ROM). The system memory 1412 may store data such as exposure and pathogen data 1407 and/or program modules such as operating system 1405 and pathogen exposure analysis software 1406 that are accessible to and/or are operated on by the one or more processors 1403.

The computer 1401 may also comprise other removable/non-removable, volatile/non-volatile computer storage media. The mass storage device 1404 may provide non-volatile storage of computer code, computer-readable instructions, data structures, program modules, and other data for the computer 1401. The mass storage device 1404 may be a hard disk, a removable magnetic disk, a removable optical disk, magnetic cassettes or other magnetic storage devices, flash memory cards, CD-ROM, digital versatile disks (DVD) or other optical storage, random access memories (RAM), read-only memories (ROM), electrically erasable programmable read-only memory (EEPROM), and the like.

Any number of program modules may be stored on the mass storage device 1404. An operating system 1405 and pathogen exposure analysis software 1406 may be stored on the mass storage device 1404. One or more of the operating system 1405 and pathogen exposure analysis software 1406 (or some combination thereof) may comprise program modules. Exposure and pathogen data 1407 may also be stored on the mass storage device 1404. In some embodiments, those program modules can embody, or can constitute, the data ingestion module 260, the factor evaluation module(s) 265, including the analysis module 266, the assessment module 270, and the output module 275 describe hereinbefore. The exposure and pathogen data 1407 can include the models 254 and the scenario data 256 described hereinbefore. The exposure and pathogen data 1407 may be stored in any of one or more databases known in the art. The databases may be centralized or distributed across multiple locations within the network 1415.

A user may enter commands and information into the computer 1401 via an input device (not shown). Such input devices comprise, but are not limited to, a keyboard, pointing device (e.g., a computer mouse, remote control), a microphone, a joystick, a scanner, tactile input devices such as gloves, and other body coverings, motion sensor, and the like These and other input devices may be connected to the one or more processors 1403 via a human-machine interface 1402 that is coupled to the bus 1413, but may be connected by other interface and bus structures, such as a parallel port, game port, an IEEE 1394 Port (also known as a Firewire port), a serial port, network adapter 1408, and/or a universal serial bus (USB).

A display device 1411 may also be connected to the bus 1413 via an interface, such as a display adapter 1409. It is contemplated that the computer 1401 may have more than one display adapter 1409 and the computer 1401 may have more than one display device 1411. A display device 1411 may be a monitor, an LCD (Liquid Crystal Display), light-emitting diode (LED) display, a television, a smart lens, a smart glass, and/or a projector. In addition to the display device 1411, other output peripheral devices may comprise components such as speakers (not shown) and a printer (not shown) which may be connected to the computer 1401 via Input/Output Interface 1410. Any step and/or result of the methods may be output (or caused to be output) in any form to an output device. Such output may be any form of visual representation, including, but not limited to, textual, graphical, animation, audio, tactile, and the like. The display device 1411 and computer 1401 may be part of one device, or separate devices.

The computer 1401 may operate in a networked environment using logical connections to one or more remote devices 1414a,b,c. In some cases, the computing 1401 can embody the risk assessment subsystem 250 (FIG. 2) and the remote devices 1414a,b,c can be embodied in, or can include, the on-premises source device(s) 220 (FIG. 2) and/or the off-premises source device(s) 230 (FIG. 2). Accordingly, in some embodiments at least one of the remote computing devices 1414a,b,c may be embodied in a personal computer, computing station (e.g., workstation), portable computer (e.g., laptop, mobile phone, tablet device), smart device (e.g., smartphone, smartwatch, activity tracker, smart apparel, smart accessory), security and/or monitoring device, a server device, a router device, a network computer, a peer device, an edge device, or other common network nodes, and so on. In addition, or in other embodiments, at least one of the remote computing devices 1414a,b,c can be embodied in, or can include, sensor devices, camera devices, a combination thereof, or the like.

Logical connections between the computer 1401 and a remote computing device 1414a,b,c may be made via a network 1415, such as a local area network (LAN) and/or a general wide area network (WAN). Such network connections may be through a network adapter 1408. A network adapter 1408 may be implemented in both wired and wireless environments. Such networking environments are conventional and commonplace in dwellings, offices, enterprise-wide computer networks, intranets, and the Internet.

Application programs and other executable program components such as the operating system 1405 are shown herein as discrete blocks, although it is recognized that such programs and components may reside at various times in different storage components of the computing device 1401, and are executed by the one or more processors 1403 of the computer 1401. An implementation of pathogen exposure analysis software 1306 may be stored on or sent across some form of computer-readable media. Any of the disclosed methods may be performed by processor-executable instructions embodied on computer-readable media.

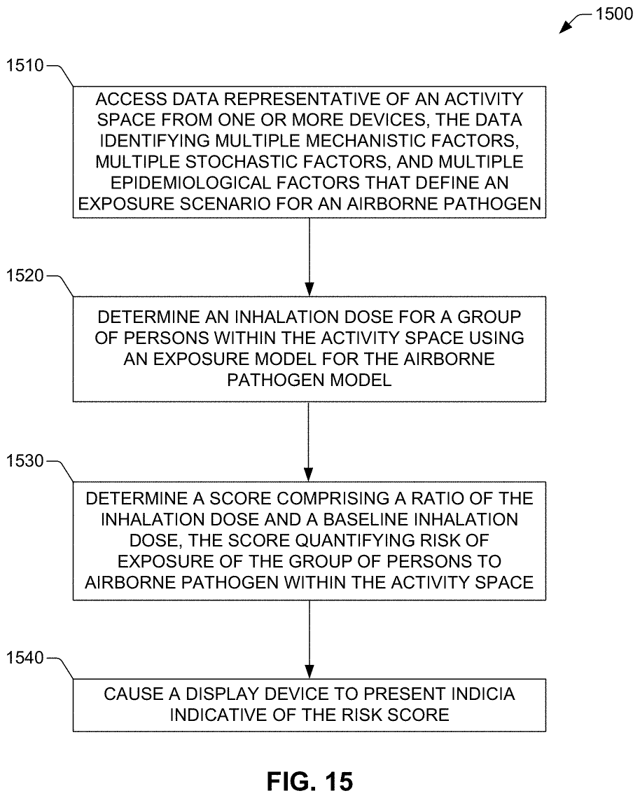

FIG. 15 is a flowchart illustrating an example method 1500 for determining inhalation dose and assessing exposure risk to airborne pathogens, in accordance with one or more embodiments of this disclosure. A computing system can perform the example method 1500 in its entirety or partially. To that end, the computing system includes computing resources that can implement at least one of the blocks included in the example method 1500. The computing resources include, for example, central processing units (CPUs), graphics processing units (GPUs), tensor processing units (TPUs), memory, disk space, incoming bandwidth, and/or outgoing bandwidth, interface(s) (such as I/O interfaces or APIs, or both); controller devices(s); power supplies; a combination of the foregoing; and/or similar resources. For instance, the computing system can include programming interface(s); an operating system; firmware; and similar resources. The computing system can embody, or can include, the risk assessment subsystem 250 (FIG. 2), in some cases. As such, in one example case, the computing system can include the risk assessment subsystem 250, at least one of the on-premises source device(s) 220 and/or at least one of the off-premises source device(s) 230. The communication architecture 240 also may be part of computing systems, in some embodiments.

At block 1510, the computing system can access data representative of an activity space from one or more devices. At least a portion of the data can identify multiple mechanistic factors, multiple stochastic factors, and multiple epidemiological factors that define an exposure scenario for an airborne pathogen. In some embodiments, at least some of the devices can be remotely located relative to the computing system. The devices can include camera devices, sensor devices, server devices, a combination thereof, or similar devices.

At block 1520, the computing system can determine an inhalation dose for a group of persons within the activity space using an exposure model for the airborne pathogen model. In some embodiments, determining the inhalation dose comprises determining an average concentration of the airborne pathogen within the activity space; and determining a product of the average concentration, an average inhalation rate, a duration of exposure to the airborne pathogen, and a number of individuals exposed to the airborne pathogen. In some cases, determining the average concentration comprises determining a near-field concentration of the airborne pathogen using eddy diffusivity, and further determining a far-field concentration of the airborne pathogen using eddy diffusivity. In addition, or in other embodiments, the determining the inhalation dose further comprises multiplying the product by a mask effectiveness parameter.

At block 1530, the computing system can determine a score comprising a ratio of the inhalation dose and a baseline inhalation dose. The score quantifies risk of exposure of the group of persons to airborne pathogen within the activity space. The baseline inhalation dose corresponds to a high-risk environment according to one of Occupational Safety and Health Administration (OSHA), Centers for Disease Control and Prevention (CDC), or an expert entity.

At block 1540, the computing system can cause a display device to present indicia indicative of the risk score. In some embodiments, the display device can be embodied in the display device 260 (FIG. 2). In some embodiments, the display device can be affixed to a fixture within the activity space (e.g., region 210 (FIG. 2)) and can be visible by occupants of the space. In other embodiments, the display device can be intergrated within a user device within the activity space.

While specific configurations have been described, it is not intended that the scope be limited to the particular configurations set forth, as the configurations herein are intended in all respects to be possible configurations rather than restrictive.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; the number or type of configurations described in the specification.

It will be apparent to those skilled in the art that various modifications and variations may be made without departing from the scope or spirit. Other configurations will be apparent to those skilled in the art from consideration of the specification and practice described herein. It is intended that the specification and described configurations be considered as exemplary only, with a true scope and spirit being indicated by the following claims.

What is claimed is:

1. An apparatus, comprising:
at least one processor; and
at least one memory device having processor-executable instructions encoded thereon that, in response to execution by the at least one processor, cause the apparatus to,
access data representative of an activity space from one or more devices, the data identifying multiple mechanistic factors, multiple stochastic factors, and multiple epidemiological factors that define an exposure scenario for an airborne pathogen;
determine an inhalation dose for a group of persons within the activity space using an exposure model for the airborne pathogen model;
determine a score comprising a ratio of the inhalation dose and a baseline inhalation dose, the score quantifying risk of exposure of the group of persons to airborne pathogen within the activity space; and
cause a display device to present indicia indicative of the score.

2. The apparatus of claim 1, wherein the inhalation dose include accumulated inhalation dose based on position over time of at least one person in the group.

3. The apparatus of claim 1, at least one of the one or more devices being remotely located relative to the apparatus and comprising one of a camera device, a sensor device, a server device, or a user device.

4. The apparatus of claim 1, the at least one memory device having further processor-executable instructions encoded thereon that, in response to execution by the at least one processor, cause the apparatus to generate, using a portion of the data, a value of a first one the multiple mechanistic factors, the multiple stochastic factors, or the multiple epidemiological factors.

5. The apparatus of claim 1, wherein determining the inhalation dose comprises,
determining an average concentration of the airborne pathogen within the activity space; and
determining a product of the average concentration, an average inhalation rate, a duration of exposure to the airborne pathogen, and a number of individuals exposed to the airborne pathogen.

6. The apparatus of claim 5, wherein the determining the inhalation dose further comprises multiplying the product by a mask effectiveness parameter.

7. The apparatus of claim 5, wherein determining the average concentration comprises determining a near-field concentration of the airborne pathogen using eddy diffusivity, and further determining a far-field concentration of the airborne pathogen using eddy diffusivity.

8. The apparatus of claim 1, wherein the baseline inhalation dose corresponds to a high-risk environment according to one of Occupational Safety and Health Administration (OSHA) or Centers for Disease Control and Prevention (CDC), or an expert entity.

9. The apparatus of claim 1, wherein the mechanistic factors comprise at least one of a number of individuals in a group within the activity space, distance between the individuals, mask effectiveness, an emission rate of infectious aerosols released through respiration, an inhalation rate, a duration of exposure, a type of activity, one or more ventilation rates, an aerosol settling rate, a pathogen degradation rate, a recirculating room filtration rate and removal efficiency, or a volume of the activity space.

10. The apparatus of claim 1, wherein the stochastic factors comprise at least one of likelihood of infectious persons present in the activity space or mask compliance within the activity space.

11. The apparatus of claim 1, wherein the epidemiological factors comprise at least one of prevalence of the airborne pathogen in a community containing the activity space, difference in variants transmission rates relative to wild type pathogen, collective immunity parameter, or impact of surveillance testing for the group.

12. The apparatus of claim 1, wherein the airborne pathogen is one of a virus, a bacterium, a fungus, or a phage that causes a disease contracted through inhalation.

13. A computer-implemented method, comprising:
accessing data representative of an activity space from one or more devices, the data identifying multiple mechanistic factors, multiple stochastic factors, and multiple epidemiological factors that define an exposure scenario for an airborne pathogen;
determining an inhalation dose for a group of persons within the activity space using an exposure model for the airborne pathogen model;
determining a score comprising a ratio of the inhalation dose and a baseline inhalation dose, the score quantifying risk of exposure of the group of persons to airborne pathogen within the activity space; and
causing a display device to present indicia indicative of the risk score.

14. The computer-implemented method of claim 13, wherein the determining the inhalation dose comprises,
determining an average concentration of the airborne pathogen within the activity space; and
determining a product of the average concentration, an average inhalation rate, a duration of exposure to the airborne pathogen, and a number of individuals exposed to the airborne pathogen.

15. The computer-implemented method of claim 13, wherein the determining the inhalation dose further comprises multiplying the product by a mask effectiveness parameter.

16. The computer-implemented method of claim 14, wherein the determining the average concentration comprises determining a near-field concentration of the airborne pathogen using eddy diffusivity, and further determining a far-field concentration of the airborne pathogen using eddy diffusivity.

17. The computer-implemented method of claim 13, wherein the baseline inhalation dose corresponds to a high-risk environment according to one of Occupational Safety and Health Administration (OSHA), Centers for Disease Control and Prevention (CDC), or an expert entity.

18. At least one computer-readable non-transitory storage medium having processor-executable instructions encoded thereon that, in response to being executed, cause an apparatus to perform operations comprising,
accessing data representative of an activity space from one or more devices, the data identifying multiple mechanistic factors, multiple stochastic factors, and multiple epidemiological factors that define a exposure scenario for an airborne pathogen;
determining an inhalation dose for a group of persons within the activity space using an exposure model for the airborne pathogen model;
determining a score comprising a ratio of the inhalation dose and a baseline inhalation dose, the score quantifying risk of exposure for a group of persons to airborne pathogen within the activity space; and
causing a display device to present indicia indicative of the risk score.

19. The at least one computer-readable non-transitory storage medium of claim 18, wherein the determining the inhalation dose comprises,
determining an average concentration of the airborne pathogen within the activity space; and
determining a product of the average concentration, an average inhalation rate, a duration of exposure to the airborne pathogen, and a number of individuals exposed to the airborne pathogen.

20. The at least one computer-readable non-transitory storage medium of claim 19, wherein the determining the inhalation dose further comprises multiplying the product by a mask effectiveness parameter.

21. The at least one computer-readable non-transitory storage medium of claim 19, wherein the determining the average concentration comprises determining a near-field concentration of the airborne pathogen using eddy diffusivity, and further determining a far-field concentration of the airborne pathogen using eddy diffusivity.
